(19) United States
(12) Patent Application Publication
(10) Pub. No.: US 2018/0160705 A1
(43) Pub. Date: Jun. 14, 2018

(54) Deacidified Juice and Method of Production Thereof

(71) Applicant: UNIVERSITÉ LAVAL, Ville de Quebec (CA)

(72) Inventors: Laurent Bazinet, L'Ancienne-Lorette (CA); Élodie Serre, Ville de Quebec (CA); Yvan Boutin, Levis (CA)

(21) Appl. No.: 15/373,303

(22) Filed: Dec. 8, 2016

(51) Int. Cl. A23L 2/02; A61K 36/45; A23L 2/70

(52) U.S. Cl. CPC A23L 2/02; A23V 2002/00; A23L 2/70; A61K 36/45

(57) ABSTRACT

Cranberry is a typical fruit from North America having a very high acidity that makes raw juice hardly acceptable for consumers. Electrodialysis with bipolar membrane (EDBM), an ecofriendly technology, allows the deacidification of cranberry juice and the production of pure acids without any chemical consumption due to the bipolar membrane in-situ generation of proton and hydroxyl species from water. Different levels of deacidification by electrodialysis was carried out to study the impact of the deacidification level on intestinal cell integrity. In the presence of deacidified cranberry juice, the integrity of Caco-2 cell monolayers measured by the ΔTEER was increased by 56% in comparison with raw cranberry juice.

### FIELD

This invention relates to a method for lowering the acid content of certain juices and juices produced thereof. Particularly, this invention also provides a deacidified cranberry juice that has less adverse effects on the intestinal epithelium.

### BACKGROUND

Cranberry is a typical fruit from North America, belonging to the family “ericaceous” and the gender “Vaccinium”. This fruit is well recognized for its beneficial effects on human health due to its high concentrations in polyphenols such as anthocyanins (galactosides and arabinosides of cyanidin, peonidin, malvidin and myricetin) and proanthocyanidins (PACs)¹. Several studies demonstrated that cranberry juice has a preventive effect against urinary tract infection and reduces ex-vivo adherence of Escherichia coli to vaginal epithelial cells^{1,2}. Proanthocyanidins present in cranberry juice could reduce the bacteria colonies in vaginal epithelial cells.^{3,4}. These compounds could also prevent gastric ulcers caused by Helicobacter pylori⁵, reduce cardiovascular risk factors⁶ and inhibit the formation of bacterial complexes in dental plaques′.

Consumers have been attracted by the health benefits attributed to cranberry juice but its very high organic acid content and low pH create side effects that limit its consumption. Hence, in clinical trials, high rates of withdrawals (around 40%) were observed after cranberry juice consumption due to undesirable effects (diarrhea, vomiting and bloating)^{1,5,8}. Organic acids responsible for the high titratable acidity of cranberry juice are citric, malic, quinic and succinic acids⁹. Quinic acid is present in many fruits and vegetables such as lemon, melon, peach, apple, red pepper and tomato but its concentration in cranberry juice is higher than in other fruits. Quinic acid is the second most important acid in concentration in the cranberry juice and is the reference compound for detecting cranberry juice adulteration¹⁹⁻¹³.

In order to respond to consumers' demand, there is a need to provide new and non-chemical process to lower the acidity of juices, particularly juice having high bioactivity, such as cranberry juice. Particularly, this process should not change its physicochemical and organoleptic properties, nor its bioactive properties, while improving its palatability.

### SUMMARY

Therefore, the present invention provides herewith a selective process to deacidify juice without changing physicochemical and organoleptic properties.

In a first aspect, there is provided a deacidified juice having a malic acid concentration decreased by at least about 20% from an untreated juice having an original malic acid concentration.

In a second aspect, there is provided a deacidified juice having a citric acid concentration decreased by at least about 20% from an untreated juice having an original citric acid concentration.

In a third aspect, there is provided a cranberry juice with a deacidification level of at least about 20% wherein the level is calculated with the equation (1):

deacidification level (in %)=(1−TA₍ₜ₎/TA₍ₜ₌₀₎)*100 (1),

wherein TA is the titratable acidity.

According to a further aspect, the is provided a cranberry juice with a titrable acidity lower than 7 g/L of citric acid monohydrate equivalents, when assessed using AOAC method no. 942.15 (2005).

In accordance with a further aspect of the invention, there is provided a use of a deacidified cranberry juice for the treatment and/or prevention of a urinary tract infection with minimal intestinal side effects, wherein the deacidified juice is as defined herein.

According to a further aspect, the invention provides a process for treating and/or preventing urinary tract infections without provoking serious intestinal side effects, comprising administering a deacidifed cranberry juice as defined in herein.

In accordance with a further aspect of the invention, there is provided a process for deacidifying a juice high in malic and/or citric acid concentration, comprising the steps of: subjecting the juice to electrodialysis with one anion-exchange membrane (AEM) having a cathode side and an anode side, and juxtaposed on both sides with a bipolar (BP) membrane according to scheme I:

for a length of time sufficient to decrease titrable acidity by at least about 20%.

According to a further aspect of the present invention, there is provided a process for the production of purified citric or malic acid, comprising the steps of: subjecting a cranberry juice to electrodialysis with one anion-exchange membrane (AEM) having a cathode side and an anode side, and juxtaposed on both sides with a bipolar (BP) membrane, the juxtaposed membranes comprising a C1 compartment positioned between an anode BP and the AEM, the C1 compartment connected to a recovery compartment according to scheme I:

for a length of time sufficient to decrease titrable acidity by at least about 20%; and recovering the acid from the recovery compartment.

According to a further aspect, the invention provides a method for diminishing the undesirable intestinal effects of a juice upon consumption by a subject, comprising submitting the juice to the aforementioned process.

In accordance with a further aspect, there is provided a method for diminishing the undesirable intestinal effects of a juice having an original citric acid concentration, comprising lowering the original citric acid concentration by at least about 20%.

In accordance with a further aspect, there is provided a method for diminishing the undesirable intestinal effects of a juice having an original malic acid concentration, comprising lowering the citric acid by at least about 20%.

Other objects, advantages and features of the present invention will become more apparent upon reading of the following non-restrictive description of preferred embodiments thereof, given by way of example only with reference to the accompanying drawings.

### DETAILED DESCRIPTION

### Description of the Figures

FIG. 1. Three different ED configurations: (a) ED2 MB configuration; C1 represents the organic acid recovery compartment (initially a KCl solution at 2 g/L) and C2, the electrode rinsing compartment (NaCl 20 g/L), (b) ED2MBUF configuration; C1 represents the organic acid recovery compartment (initially a citrate solution at 15 g/L) and C2, the electrode rinsing compartment (NaCl 20 g/L) and (c) EDUF configuration; C1 represents the organic acid recovery compartment (KCl 2 g/L) and C2, the electrode rinsing compartment (NaCl 20 g/L).

FIG. 2. Titratable acidity (g/L of citric acid monohydrate equivalents), in cranberry juice (black bars) and recovery solution (grey bars), in three different ED configurations tested a) ED2 MB, b) ED2MBUF, c) EDUF.

FIG. 3. pH of a) cranberry juice, and b) recovery solution during the ED deacidification processing using three different cell configurations (ED2 MB, ED2MBUF, EDUF).

FIG. 4. Evolution of conductivity (mS/cm) in a) cranberry juice and b) recovery solution during the ED deacidification processing using the three different cell configurations (ED2 MB, ED2MBUF, EDUF).

FIG. 5. Evolution of a) quinic, b) citric, c) malic and d) succinic acid concentrations (in mg/L) in cranberry juice (black dots) and in recovery solutions (white triangles), during the ED treatment with the ED2 MB configuration.

FIG. 6. Ionic fractions of a) quinic acid b) citric acid and c) malic acid.

FIG. 7. Global system resistance of three configurations tested (ED2 MB, ED2MBUF, EDUF) for cranberry juice deacidification.

FIG. 8. Protocol for electrodialysis and digestion of deacidified juice prior to testing in Caco-2 cells.

FIG. 9. Co-culture system constructed with Caco-2 cell monolayers and human leukemia monocytic cell line (THP-1 cells). Deacidified cranberry juice was added to the apical compartment of this co-culture system.

FIG. 10. Effect of different deacidification levels of cranberry juice on the variation in TransEpithelial Electrical Resistance (ΔTEER) (Ω*cm2) of Caco-2 monolayers (* P<0.05, ** P<0.01, *** P<0.001, n.s P>0.05). HBSS is Hank's Balanced Salt Solution and S.E.M corresponds to Standard Error of the Mean.

FIG. 11. Titratable acidity (g/L of citric acid monohydrate equivalents), in grapefruit juice (black bars) and recovery solution (grey bars), in ED2 MB.

FIG. 12. Titratable acidity (g/L of citric acid monohydrate equivalents), in lemon juice (black bars) and recovery solution (grey bars), in ED2 MB.

FIG. 13. Evolution of malic acid concentrations (in mg/L) in lemon juice (triangles) and in recovery solutions (dots), during the ED treatment with the ED2 MB configuration.

FIG. 14. Evolution of citric acid concentrations (in mg/L) in lemon juice (triangles) and in recovery solutions (dots), during the ED treatment with the ED2 MB configuration.

FIG. 15. Evolution of malic acid concentrations (in mg/L) in grapefruit juice (triangles) and in recovery solutions (dots), during the ED treatment with the ED2 MB configuration.

FIG. 16. Evolution of citric acid concentrations (in mg/L) in grapefruit juice (triangles) and in recovery solutions (dots), during the ED treatment with the ED2 MB configuration.

### ABBREVIATIONS AND DEFINITIONS

### Abbreviations

ED, Electrodialysis; TEER, TransEpithelial Electrical Resistance; EDBM, Electrodialysis with Bipolar Membrane; HBSS, Hank's Balanced Salt Solution; RPMI, Roswell Park Memorial Institute medium; PACs, ProAnthoCyanidins; TA, Titratable Acidity.

### Definitions

The term “about” as used herein refers to a margin of + or −10% of the number indicated. For the sake of precision, the term about when used in conjunction with, for example: 90% means 90%+1-9% i.e. from 81% to 99%. More precisely, the term about refer to + or −5% of the number indicated, where for example: 90% means 90%+1-4.5% i.e. from 86.5% to 94.5%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

### DETAILED DESCRIPTION OF PARTICULAR EMBODIMENTS

In order to respond to consumers' demand, a selective process to deacidify cranberry juice without changing its physicochemical and organoleptic properties has been developed. After deacidification, the content of certain organic acids is low enough to provide a juice with a mellow taste without addition of sweetener. Surprisingly, it has now been demonstrated that this deacidified juice has less adverse effects on the intestinal epithelium than untreated (i.e. “native”) cranberry juice.

### Juice Composition

In accordance with a particular embodiment, the invention provides a deacidified juice having a malic acid concentration decreased by at least about 23% from an untreated juice having an original malic acid concentration. Particularly, the malic acid is 40%, more particularly 60%, or most particularly 80% lower than in the untreated juice.

In accordance with an alternative embodiment, there is provided a deacidified juice having a citric acid concentration decreased by at least about 25% from an untreated juice having an original citric acid concentration. Particularly, the citric acid is 40%, more particularly 50%, most particularly 60% lower than in the untreated juice.

According to a particular embodiment of the invention, the juice is a fruit or a vegetable juice, high in acidity. Particularly, the juice is a fruit juice. More particularly, the juice is made from a fruit selected from the group consisting of: lemon, lime, grapefruit, passion fruit, apple, cherries, loganberry, plum, prune, pomegranate, pear, blackberry, raspberry, strawberry, tangerine blueberry, currant, blackcurrant, pineapple, kiwi, peach, apricot, tomato, orange, acai and cranberry juice. Most particularly, the juice is selected from the group consisting of: lemon, lime, grapefruit, and cranberry juice. Still, most particularly, the juice is cranberry juice.

In accordance with a particular embodiment, the invention provides a deacidified cranberry juice. Particularly, the deacidified cranberry juice has a citric acid concentration lower than about 16,200 mg/L, more particularly lower than about 12,000 mg/L, still more particularly lower than about 10,000 mg/L, most particularly lower than about 8,000 mg/L, and still most particularly lower than about 6,000 mg/L.

In accordance with an alternative embodiment, the invention provides a cranberry juice having a malic acid concentration lower than about 10,600 mg/L, particularly lower than about 10,000 mg/L, more particularly lower than about 8,000 mg/L, still more particularly lower than about 6,000 mg/L, most particularly lower than about 4,000 mg/L, and still most particularly lower than about 2,000 mg/L.

According to an alternative embodiment, the invention provides a cranberry juice with a deacidification level of at least about 20% wherein the level is calculated with the equation (1): deacidification level (in %)=(1−TA₍ₜ₎/TA₍ₜ₌₀₎)*100 (1), wherein TA is the titratable acidity. Particularly, the deacidification level of the cranberry juice is at least about 25%, more particularly at least about 30%, 35%, 37%, 40%, 45%, 50%, 55%, 60%, 65%, 70%, 75% or still most particularly at least about 77%.

In according to a further embodiment, the invention provides a cranberry juice with a titrable acidity lower than 7 g/L of citric acid monohydrate equivalents, when assessed using AOAC method no. 942.15 (2005). Particularly, the titrable acidity is lower than about 5 g/L, and more particularly lower than about 3 g/L.

According to an alternative embodiment, the invention provides the deacidified juice as defined herein wherein the resulting pH is about the same as the original (i.e. untreated) juice's pH. In particular, the treated juice pH has less than about 0.5 unit difference from the original juice's pH, more particularly less than about 0.3 pH unit.

According to a further embodiment of the invention, the resulting deacidified juice is easier to drink than the untreated juice and does not require addition of sweeteners to mellow out the taste. Still, particularly, the mellow-tasting juice of the invention substantially retains the original bioactive molecules that are sought after for providing health benefits to the consumer.

In accordance with a further embodiment of the invention, the deacidified juice has substantially the same amount of sugar as its original untreated version.

In accordance with a further embodiment of the invention, the deacidified juice has substantially the same amount of quinic acid as its original untreated version.

In accordance with a further embodiment of the invention, the deacidified juice has substantially the same amount of bioactive molecules, such as proanthocyanidins or anthocyanins, as its original untreated version.

### Process

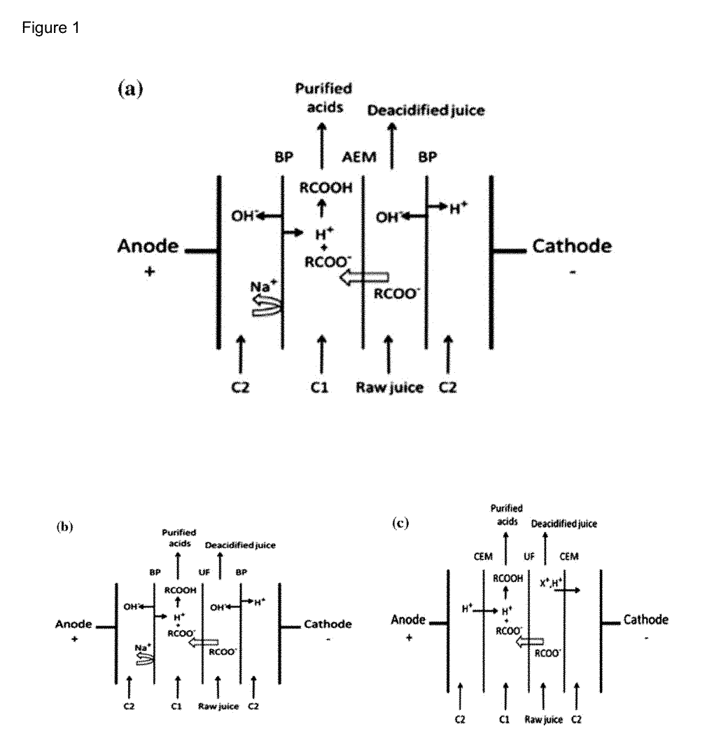

In accordance with a further embodiment of the invention, there is provided a process for deacidifying a juice high in malic and/or citric acid concentration, comprising the steps of: subjecting the cranberry juice to electrodialysis with one anion-exchange membrane (AEM) having a cathode side and an anode side, and juxtaposed on both sides with a bipolar (BP) membrane according to FIG. 1.

for a length of time sufficient to decrease titrable acidity by at least about 20%.

Particularly, within the context of the aforementioned process, the electrodialysis is performed at room temperature under a constant electric field of 10 V for a duration of at least about 20 minutes, but more particularly for a maximum duration of about 3 hours.

More particularly, within the context of the aforementioned process, the original quinic acid concentration is substantially maintained after the process steps.

More particularly, within the context of the aforementioned process, the original sugar concentration is substantially maintained after the process steps.

More particularly, within the context of the aforementioned process, the original concentrations of bioactive molecules are substantially maintained after the process steps. Still more particularly, the bioactive molecules are polyphenolic molecules, such as for example, proanthocyanins (PACs) or anthocyanins.<

More particularly, and surprisingly, the process of the invention yields a deacidified juice where the integrity of the bioactive molecules is maintained. In fact, such molecules as PACs or anthocyanins are easily oxidized molecules, and one could have thought that such a process could have affected their integrity, which is not the case here.

In accordance with an alternative embodiment, the invention provides a process for the production of purified citric or malic acid, comprising the steps of: subjecting the cranberry juice to electrodialysis with one anion-exchange membrane (AEM) having a cathode side and an anode side, and juxtaposed on both sides with a bipolar (BP) membrane, the juxtaposed membranes comprising a C1 compartment positioned between an anode BP and the AEM, the C1 compartment connected to a recovery compartment according to scheme I (above); for a length of time sufficient to decrease titrable acidity by at least about 20%; and recovering the acid from the recovery compartment.

### Uses and Methods of Use

In accordance with a further embodiment, the invention provides use of a deacidified cranberry juice for the treatment and/or prevention of a urinary tract infection with minimal intestinal side effects, wherein the deacidified juice is as defined herein.

In accordance with an alternative embodiment, the invention provides a method for treating and/or preventing urinary tract infections without provoking serious intestinal side effects, comprising administering a deacidifed cranberry juice as defined herein.

In accordance with an alternative embodiment, the invention provides a method for diminishing the undesirable intestinal effects of a juice upon consumption by a subject, comprising submitting the juice to the process as defined herein.

In accordance with an alternative embodiment, the invention provides a method for diminishing the undesirable intestinal effects of a juice having an original citric acid concentration, comprising lowering the original citric acid concentration by at least about 25%. Particularly, the citric acid concentration is lowered according to the process as defined herein.

In accordance with an alternative embodiment, the invention provides a method for diminishing the undesirable intestinal effects of a juice having an original malic acid concentration, comprising lowering the citric acid by at least about 23%. Particularly, the malic acid concentration is lowered in accordance to the process as defined herein.

According to a further embodiment of the invention, the resulting deacidified juice is easier to drink than the untreated juice and does not require addition of sweeteners to mellow out the taste before its use as always used in the past by the consumer. Still, particularly, the consumption of the mellow-tasting juice of the invention avoids undesirable intestinal side effects while substantially retaining the original bioactive molecules that are sought after for providing health benefits to the consumer. In particular, the health benefits for cranberry juice are well documented and may provide protection and help prevent urinary tract infections, dental cavities, cardiovascular disease, carcinogenesis, help maintain a healthy microbiota. Other known health benefits of cranberry juice include relief of urinary tract infection, respiratory disorders, kidney stones, cancer, and heart disease. It is also beneficial in preventing stomach disorders and diabetes, as well as gum diseases caused by dental plaque. Cranberry juice contains anti-oxidants which prevent cell damage. Consuming cranberry juice can result in enhancing the memory function, preventing the anti-aging process, avoiding inflammation of the body parts, discouraging formation of kidney stones, counter-attacking the cancer-inducing compounds and controlling obesity.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Examples

### Example 1—Deacidification of Cranberry Juice by Electrodialysis: Impact of Membrane Types and Configurations on Acid Migration and Juice Physicochemical Characteristics

### Materials and Methods

### Cranberry Juice

The clarified and pasteurized cranberry juice was obtained from fresh fruits (Fruit d'Or, Notre-Dame-de-Lourdes, Quebec, Canada). The juice was stored at −20° C. and thawed at 4° C. before each experiment. The physicochemical characteristics of the cranberry juice used for the experiment are presented in Table 1.

TABLE 1
Physicochemical characteristics of the clarified
and pasteurized cranberry juice.

| | |
|---|---|
| pH | 2.4 ± 0.1 |
| Conductivity (mS/cm) | 2.8 ± 0.2 |
| Titratable acidity ( g/L of citric | 9.5 ± 1.7 |
| acid monohydrate equivalents ) | |
| Total soluble solids (° Brix) | 7.0 ± 0.5 |
| Colorimetry | |
| L* | 27.1 ± 0.2 |
| a* | 1.5 ± 0.4 |
| b* | −0.4 ± 0.1 |
| Total proanthocyanidins (mg/L) | 106 ± 11 |
| Total polyphenols (mg equivalent | 680 ± 70 |
| gallic acid/L) | |
| Anthocyanin contents (mg/L) | |
| Cyanidin-3-galactose | 13.8 ± 1.2 |
| Cyanidin-3-glucose | 0.3 ± 0.0 |
| Cyanidin-3-arabinose | 14.4 ± 1.3 |
| Peonidin-3-galactose | 20.0 ± 1.9 |
| Peonidin-3-glucose | 1.8 ± 0.2 |
| Peonidin-3-arabinose | 10.8 ± 1.0 |
| Organic acid contents (mg/L) | |
| Citric acid | 9 800 ± 800 |
| Quinic acid | 8 200 ± 300 |
| Malic acid | 5000 ± 500 |
| Succinic acid | 7 600 ± 1000 |

### Electrodialytic Configurations:

Electrodialysis experiments were performed using a MP type cell (ElectroCell AB, Taby, Sweden) with an effective surface area of 100 cm². Three different ED configurations were tested (FIG. 1).

ED2 MB:

In this configuration, two compartments (raw juice and organic acids recovery compartments) were formed by stacking two bipolar membranes (BP-1, Tokuyama Soda Ltd., Tokyo, Japan) and one food grade Neosepta anion-exchange membrane (AMX-SB, Tokuyama Soda Ltd., Tokyo, Japan) (FIG. 1a). Bipolar membranes allow the formation of organic acid through the production of H⁺ in C1 compartment while the anionic membrane allows the migration of organic acids according to their pKa.

ED2MBUF:

In comparison with the previous configuration, the anion-exchange membrane (AEM) was replaced by a polysulfone ultrafiltration membrane (UF) with a molecular weight cut off of 3 kDa (GE, Polysulfone, France) (FIG. 1b). The UF membrane would facilitate the migration of organic acid due to its large cut off, so theoretically the desired acidification level can be reached more quickly.

EDUF:

In this configuration, the two bipolar membranes of configuration ED2MBUF were replaced by cation-exchange membranes (CMX-SB, Tokuyama Soda Ltd., Tokyo, Japan) (FIG. 1c).

For each configuration, the cell had three closed loops, connected to separate external reservoirs and allowing recirculation of the three solutions (acid recovery, cranberry juice and electrode rinsing solutions) during treatment. The solutions were circulated using three centrifugal pumps and the flow rates controlled by flow-meters (Aalborg Instruments and Controls, Inc., Orangeburg, USA). The anode was a dimensionally stable electrode (DSA-O₂) and the cathode was a food grade stainless steel electrode. The anode/cathode voltage difference was supplied by an electrical power supply (Model HPD 30-10, Xantrex, Burnaby, Canada).

### Protocol

A 20 g/L NaCl solution was circulated in the electrode rinsing compartments of all three configurations but a 2 g/L KCl solution was used in the recovery compartment of the ED2 MB and EDUF configurations. In ED2MBUF configuration, a 15 g/L citrate solution was used in the recovery compartment instead of KCl to avoid migration of potassium in the cranberry juice through UF membrane which could have a negative effect on juice composition and flavor. Cranberry juice and KCl/citrate solutions flow rates were both maintained constant at 400 mL/min whereas a flow rate of 450 mL/min was used for the NaCl solution. The volume of cranberry juice and KCl solutions were 800 mL whereas for electrolyte solution it was 1 L and the volume remained constant during experiment. Treatments were performed at room temperature under a constant electric field of 10 V corresponding respectively to averaged current densities of 38, 10 and 11 A/m² for ED2 MB, ED2MBUF and EDUF configurations. The current density for ED2MBUF and EDUF configurations is four times lower than ED2 MB configuration since ultrafiltration membranes are less conductive than ionic exchange membrane leading to a lower current density. The treatment duration was set for 3 hours except for the ED2 MB configuration which was prolonged to 6 hours, to determine the full potential of this configuration. In all configurations, three replicates were performed. The electrical conductivity and the pH of both the cranberry juice and the recovery solution were recorded every hour. The thickness and electrical conductivity of membranes were measured before and after each treatment. In external reservoirs, samples (15 mL) of recovery solution and cranberry juice were collected every hour and analyzed to characterize the evolution of their various physicochemical characteristics (pH, conductivity, titratable acidity, total soluble solids, juice color and mineral ion concentration). For the ED2 MB configuration only, additional analyses were performed on each cranberry juice and recovery solution for anthocyanin, proanthocyanidin, organic acid and total polyphenol contents determination. After each experiment, samples were stored at −20° C. and thawed at 4° C. before analysis.

### Analyses

### Physico-Chemical Characteristics of Cranberry Juice and Acid Recovery Solutions:

pH:

The pH of both the acid recovery solutions and the cranberry juice was measured using a pH-meter model SP20 (VWR Symphony, Thermo Orion West Chester, Pa., USA).

### Conductivity:

An YSI conductivity meter (Model 3100, Yellow Springs Instrument, Yellow Springs Ohio, USA) equipped with a YSI immersion probe (Model 3252, cell constant K=1 cm⁻¹) was used for recording the acid recovery solution and the juice conductivities.

### Titratable Acidity:

All along the treatments, the titratable acidity of both the recovery solution and the cranberry juice was determined by titrating 4 mL of these solutions in 40 mL of degassed distilled water with a 0.1 M NaOH solution until pH 8.2 was reached (AOAC method no. 942.15.). The titratable acidity was expressed as g equivalent citric acid monohydrate per L of product.

### Total Soluble Solids:

The total soluble solid content of both the recovery and juice solutions was determined using a refractometer Reichert AR 200 (Reichert Inc., Depew, N.Y., USA).

### Juice Color:

A chromometer (Model Minolta meter CR-300, Konica Minolta Inc., Mississauga, ON, Canada) was used to determine the color parameters of both the cranberry juice and the recovery solution. Results were expressed as L* (luminescence or lightness), a*(intensity of color varying from red to green) and b* (intensity of color varying from yellow to blue)¹⁸.

### Ca, K, Na, Mg and P Concentration Determination:

Mineral concentrations were determined by ICP (ICP-OES, Optima 4300, Dual View, Perking Elmer, Shelton, Conn., USA). The wavelengths used to determine each element were: 766.490 nm—Potassium, 589.592 nm—Sodium, 178.221 nm—Phosphorus, 279.077 nm—Magnesium, 317.933 nm—Calcium. The analyses for all ions were carried out in radial view. The Quickchem method 12-115-01-1-A and Quickchem method 10-117-07-1-C were used for the determination of P/PO₄. Samples (10 mL, diluted 1:5 in distilled water) were collected during the process and used for ion migration determination.

### Anthocyanin Content:

Individual anthocyanin composition of both the recovery solution and the cranberry juice were analyzed by HPLC¹⁹ using an Agilent 1100 series (Agilent technologies) system equipped with a diode array detector. Briefly, 0.5 mL of samples were injected and anthocyanin were analyzed with a Luna 5 um C₁₈ column (2*250 mm, Phenomenex, Torrance, Calif., USA). Solvent A, 100% acetonitrile and solvent B, acetic acid/acetonitrile/phosphoric acid (10%/5%/1%) in water were used for elution at 1 mL/min. The detection wavelength was 520 nm. Anthocyanins were quantified in mg/L of cyanidin-3-glucose equivalents, using the molar extinction coefficient of 26.900 and the molecular weight of 4 492 g/mol.

### Proanthocyanidin Content:

The proanthocyanidin profile was determined using an Agilent 1100 series HPLC system equipped with a fluorescence detector (Waters, model 474, Milford, Calif., USA) according to the method of Khanal et al. (2009)²⁰. Briefly, samples (0.5 mL) were injected on a Luna 5 μm silica column (3*150 mm, Phenomenex, Torrance, Calif., USA) and proanthocyanidins were separated according to their degree of polymerization, using a linear gradient from 0% to 40% B, in 35 min; 40% to 100% B, in 40 min; 100% isocratic B, in 45 min; and 100% to 0% B, in 50 min. Solvent A (0.65 mL/min) was dichloromethane/methanol/acetic acid/water (82%/14%/2%/2%), and solvent B (0.65 mL/min) was methanol/acetic acid/water (96%/2%/2%). Emission and excitation wavelength were set at 316 and 276 nm, respectively. Proanthocyanidins of different degrees of polymerization were quantified using a calibration curve of epicatechin. A correction factor was used to comply with the different responses factors of monomeric to polymeric proanthocyanidins. The content of each proanthocyanidin was expressed as mg/L epicatechin equivalents.

### Organic Acid Content:

Before analysis, organic acids from both the recovery solution and the cranberry juice were extracted by SPE using C18 cartridges (non end-capped 6 mL, 500 mg, Silicycle, Québec City, Qc, Canada). The cartridges were first conditioned with methanol (5 mL), and washed with distilled water (5 mL) and 10 mL of acetonitrile/water (50 v/v) solution. After the cartridges were dried, 10 mL of samples were dropped off and only 5 mL were used for HPLC analysis.

Organic acids were analyzed by HPLC using a Waters system (Milford, Mass., USA) equipped with an UV detector (Waters, model 966), according to the AOAC method no. 986.13. Briefly, organic acids were separated on a Synergi Hydro-RP column (250 mm*4 mm, Phenomenex, Torrance, Calif., USA) using a mobile phase composed of a 0.2 M (v/v) KH₂PO₄ (pH 2.4) solution at flow rate of 0.8 mL/min. The detection wavelength was set at 214 nm. Malic, citric, quinic and succinic acids were identified and quantified using the retention times and calibration curves of authentic standards (Sigma Company, Saint Louis, Mo., USA).

### Total Polyphenols:

The total polyphenols concentration was determined using the Folin-Ciocalteau assay as microscaled by Waterhouse²¹, using a UV-Visible spectrometer (Agilent Technologies, Palo Alto, Calif., USA). The detection wavelength was set at 765 nm. Concentration in total polyphenols was expressed in mg/L of gallic acid equivalents.

### Electrodialytic Parameters and Membrane Characteristics

### Global System Resistance:

The global system resistance (R) was calculated using Ohm's law (U/=R×I) The voltage (U) was directly obtained from the power supply whereas the resulting current intensity (I) was read on a Mastercraft numerical multimeter (Model 52-0060-2, Mastercraft, Toronto, Canada).

### Membrane Thickness:

The membrane thickness was measured using an electronic Digital Micrometer (Marathon Watch Company LTD., Richmond Hill, ON, CA). Before and after each treatment, the membrane thickness value was measured and averaged from six measurements at different locations on the membrane. This value was expressed as cm and allowed calculating the conductivity of membranes (mS/cm) according to the following equation:

$$K = \frac{e}{Rm * A}$$

where e is the membrane thickness (cm), Rₘ, the transversal resistance of the membrane (Q), and A, the electrode area (1 cm²).

### Statistical Analyses:

An analysis of variance (ANOVA) was performed on data using SAS software (version 6.3 for windows, SAS Institute Inc., Cary, N.C., USA). Treatments were compared using Tukey's test at α=0.05.

### Results & Discussion

### Cranberry Juice and Recovery Solution Analysis

### Titratable Acidity:

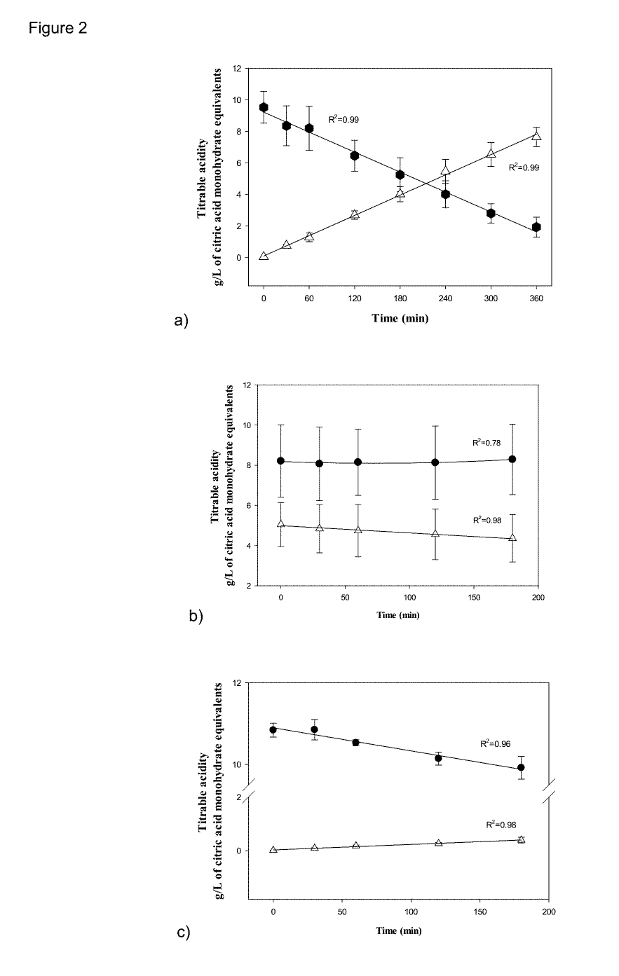

The titratable acidity of both the treated juice and the recovery solution varied significantly among the three configurations (P<0.05) (FIG. 2). In ED2 MB configuration, the titratable acidity of the treated cranberry juice decreased from 9.52±1.0 to 1.92±0.6 g/L of citric acid monohydrate equivalents (P<0.05) after six hours of treatment while it increased from 0.03±0.0 to 7.63±0.6 g/L of citric acid monohydrate equivalents in the recovery solution (P<0.05) during the same period. In this configuration, the decrease in juice acidity corresponded to a deacidification of 40% after 3 hours, and of 80% after six hours, for a deacidification level of 0.22%/min. Similar results were observed with the EDUF configuration but the deacidification level was considerably slower (0.047%/min); resulting in significantly lower level of deacidification in cranberry juice (8.5%; from 10.8±0.2 to 9.9±0.3 mL) after three hours of treatment. In contrast, the titratable acidity of the juice remained constant at all time in the ED2MBUF configuration, and the deacidification rate was null (0%; P=0.56).

It was important to mention that both the ED2 MB and the EDUF configurations successfully deacidified cranberry juice but at different levels: 40% vs 8% after three hours of treatment, respectively. The difference between these configurations is the presence of the bipolar membrane. By splitting water, bipolar membrane provided negatively charged OH⁻ molecules in the cranberry juice, which allowed modifying its pH and may have favored the migration of organic acids. The production of OH⁻ can increase the concentration of dissociated, and consequently charged, organic acids according to their pKa. To confirm this hypothesis, two experiments were performed (NaOH solution (8 g/L) or NaCL solution (8 g/L) in the C2 compartments in ED3C configuration by Vera et al. (2007) to confirm that the presence of OH⁻ in cranberry juice is necessary to obtain optimal rate of deacidification. This configuration was the same as the one used by Vera et al (2007)¹⁵ (ED3C) on tropical fruit juice and the electrodialysis parameters were the same as those used in the present study except for the voltage which was controlled at 7V (instead of 10 V) to avoid reaching the limiting current density (LCD) with this special configuration; the LCD was measured according to the Cowan and Brown method²² and was equal to 9.5V. After one hour of treatment, the titratable acidity in NaOH configuration decreased from 9.0±0.1 to 7.6±0.4 g/L of citric acid monohydrate equivalents (P<0.05) in the cranberry juice while in NaCl configuration it remained constant around 8.3±0.5 g/L of citric acid monohydrate equivalents (P=0.13). With the NaOH solution, the deacidification was around 15% after one hour of treatment whereas in NaCl solution the deacidification was null. Finally, the ED2 MB configuration was the most efficient in reducing the titratable acidity of cranberry juice, within the shortest time, as the deacidification level was more than five times faster in this configuration in comparison with other systems, including the EDUF configuration.

pH:

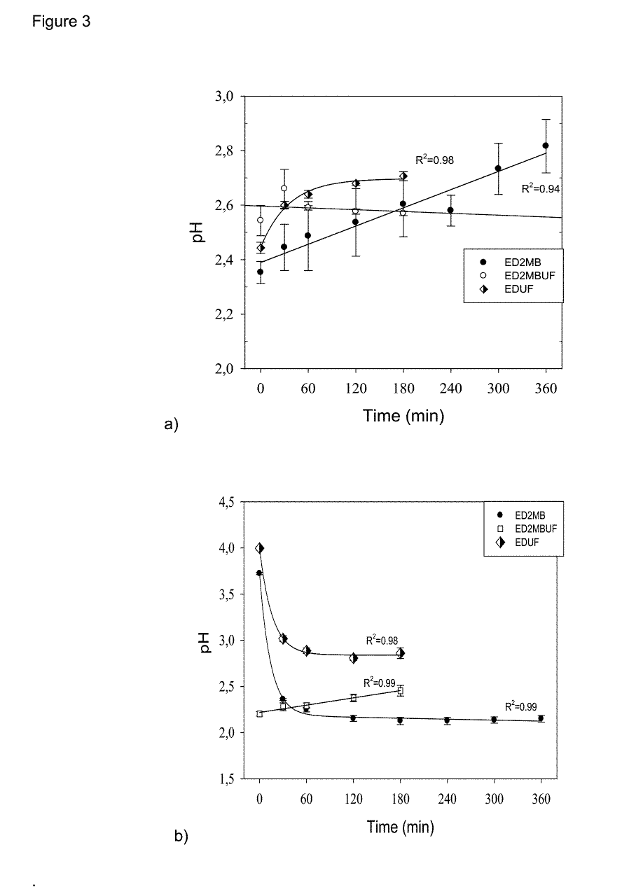

Cell configuration significantly impacted the evolution of pH in both the cranberry juice and the recovery solution, as significant changes in pH of both solutions were observed in the ED2 MB configuration (P<0.05) and in the EDUF configuration (P<0.05) but not in the ED2MBUF configuration (P>0.05) (FIG. 3).

Indeed, in ED2 MB configuration, the pH of the juice increased from 2.35±0.04 to 2.82±0.09 (P<0.05), whereas, in the recovery compartment, it decreased from 3.72±0.01 to 2.15±0.04 all along this experiment (P<0.05). In contrast, in the ED2MBUF configuration, no significant change in pH evolution (no significant change in deacidification level) occurred in both solutions (P>0.05). One hypothese may be proposed: the H+ ions, generated by the bipolar membrane on the anode side, could quickly migrate to the juice through the ultrafiltration membrane and neutralize the negatively charged organic acids of the juice, therefore stopping their migration. In EDUF configuration, pH showed a similar evolution to that observed in ED2 MB configuration: in the cranberry juice, pH increased from 2.44±0.02 to 2.70±0.02 (P<0.05) while it decreased from 3.99±0.02 to 2.86±0.06 in the recovery solution (P<0.05).

### Conductivity:

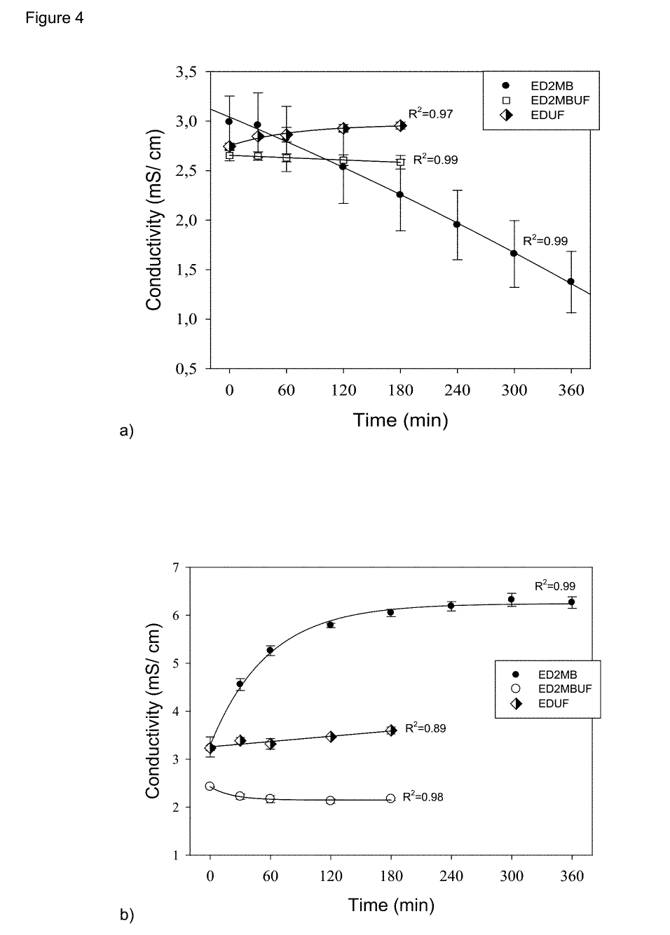

The analysis of variance showed that cell configuration had a significant impact on the evolution of conductivity in both juice and recovery compartments (P<0.05) (FIG. 4).

In ED2 MB configuration, the conductivity of juice decreased significantly from 2.99±0.30 to 2.25±0.40 mS/cm (P<0.05) after three hours, and reached 1.37±0.30 mS/cm after six hours hence showing a demineralization rate of 0.004 mS/cm·min. In the same time, the conductivity measured in the recovery compartment increased from 3.25±0.20 to 6.04±0.08 mS/cm after three hours, and reached 6.3±0.10 mS/cm, after six hours of treatment (P<0.05). This evolution can be related to the production of H⁺ by the bipolar membrane and the fact that there were some leakages or migration of potassium through the bipolar membrane (as explained later). In a previous study¹⁵, aiming at deacidifying tropical juice using a bipolar membrane, titratable acidity, pH and conductivity evolved in a similar manner as our results, but the authors did not explained such variation. Current densities applied in tropical juices (50 to 400 A·m⁻²) allowed the deacidification of tropical juice up to 70%, while increasing the pH from 2.9 to 4.0, but the duration of the treatment was not mentioned. In the present study, the deacidification level of cranberry juice reached 80% after six hours of treatment, with little variation in juice pH (from 2.4 to 2.8), using a current density of only 37.6 A·m⁻². In EDUF configuration, the conductivity increased from 2.74±0.03 to 2.95±0.03 mS/cm (mineralization rate of 0.03 mS/cm·min) (P<0.05), and from 3.23±0.05 to 3.6±0.07 mS/cm (P<0.05) in the cranberry juice and the recovery solution respectively, after three hours of treatment. In the ED2MBUF configuration, the conductivity of the cranberry juice remained constant to its initial value of 2.6 mS/cm during the whole process (P>0.05) while it decreased slowly from 2.46±0.01 to 2.16±0.03 mS/cm in the recovery compartment.

The significant decrease in the conductivity of cranberry juice in ED2 MB configuration could be explained by the migration of organic acids or/and other charged molecules and mineral ions (mineral ions: K⁺ as explained later) during ED process. This is consistent with the increase of conductivity observed in the recovery solution. The low change in conductivity observed in EDUF configuration could be related to the low migration rate of organic acids, as also suggested by the low changes observed in juice titratable acidity, and/or to the migration of cations from the recovery compartment. In ED2MBUF configuration, no variation in conductivity as well as no change in titratable acidity was observed in the cranberry juice since cations or protons electrogenerated by the bipolar membrane in the recovery solution potentially balanced the conductivity. As such, the migration of electrogenerated protons through the UF membrane could efficiently neutralize the charge of juice organic acids, therefore stopping their migration towards the anode, and into the recovery compartment, through the UF membrane. Consequently, ED2MBUF configuration is unsuitable for the deacidification of cranberry juice.

### Total Soluble Solids:

In ED2 MB configuration, the total soluble solids in cranberry juice decreased significantly from 6.76±0.02 to 5.06±0.01 (P<0.05) whereas it increased from 0.11±0.02 to 1.86±0.01 (P<0.05) in the recovery solution. In contrast, the level of total soluble solids remained unchanged (P=0.06) in the juice and recovery solution issued from the EDUF and ED2MBUF configurations.

Degree Brix is a measurement of the total soluble solids in solution (particularly minerals, sugars and organic acids). In ED2 MB configuration, the decrease in degree brix of the cranberry juice during the treatment is likely attributable to a significant migration of organic acids as well as some minerals. This was confirmed by the changes observed in the titratable level of the juice solution issued from this configuration. Also, such evolution of degree brix has already been reported in studies on deacidification of tropical juices¹⁵.

### Mineral Ion Concentration:

All tested configurations induced significant variations in the mineral ion concentration of both the cranberry juice and the recovery solution (Table 2). Within six hours of treatment, the concentration of potassium ion decreased significantly from 760±8.0 to 320±90 ppm in cranberry juice treated with ED2 MB configuration, while it increased significantly from 840±30 to 1650±220 ppm in the recovery solution. At the end of the first three hours, the rate of potassium ions migration from cranberry juice to the recovery compartment was 0.8 ppm/min, but it increased to 1.6 ppm/min thereafter. However, sodium, calcium and magnesium ions concentration remained constant all along the experiment (P>0.05). Indeed, the migration rate of potassium, the only migrating mineral cation, decreased in cranberry juice and its concentration consequently increased in the recovery compartment. The migration of this ion is explained by its high electrophoretic mobility and the fact that it is the most abundant ion in cranberry juice. Despite their low concentrations, phosphorus ions decreased significantly in cranberry juice and were mostly retrieved in recovery solution. The ED2MBUF configuration caused a significant decrease in potassium concentration of cranberry juice, which decreased from 600±40 to 340±40 ppm at a rate of 1.5 ppm/min, whereas a significant increase in the recovery solution was observed from 840±30 to 1650±220 ppm at a rate of 4.5 ppm/min. The concentration of sodium ions also increased significantly in both the cranberry juice and the recovery solution. Sodium ions are present in the electrode rinsing solution, therefore these ions passed through bipolar membranes. Ultrafiltration membranes have larger pore than ionic membrane, hence all mineral ions (sodium, calcium, magnesium, potassium and phosphorus ions) can easily migrate through these membranes. The evolution of mineral ions concentrations in both compartments of the EDUF configuration was very different from the others tested configurations; the potassium ion concentration remained constant all along the treatment in the cranberry juice at 670±150 ppm. However, the calcium and sodium ions migrated significantly through the cation-exchange and the ultrafiltration membranes, which increased their total concentration from 120 ppm to 640 ppm in the recovery solution. Positively charged minerals migrated from one compartment to another, which resulted in a continuous recirculation of these ions.

TABLE 2
Evolution of mineral contents (in mg/L) in cranberry juice and recovery solution
during treatment with the ED2BM, ED2MUF and EDUF configuration.

| | | Calcium | | Potassium | | Magnesium | |
|---|---|---|---|---|---|---|---|
| | Time | Cranberry | Recovery | Cranberry | Recovery | Cranberry | Recovery |
| | (min) | juice | solution | juice | solution | juice | solution |
| ED2MB | 0 | 69.7 ± 10.0^{a} | 0.4 ± 0.0^{a} | 759.9 ± 8.0^{a,b} | 835.9 ± 34.2^{a} | 53.1 ± 8.0^{a} | 0.0 ± 0.0^{a} |
| configuration | | | | | | | |
| | 60 | 82.7 ± 3.2^{a} | 0.7 ± 0.1^{a,b} | 830.0 ± 48.7^{a} | 1 141.5 ± 125.5^{a,b} | 63.5 ± 2.3^{a} | 0.0 ± 0.0^{a} |
| | 120 | 77.3 ± 5.1^{a} | 0.6 ± 0.2^{a,b} | 734.6 ± 73.1^{a,b} | 1 007.4 ± 380.7^{a,b} | 58.9 ± 2.8^{a} | 0.0 ± 0.0^{a} |
| | 180 | 71.7 ± 6.5^{a} | 0.9 ± 0.2^{a,b} | 619.6 ± 80.9^{a,b,c} | 1 508.8 ± 199.5^{a,b} | 53.2 ± 4.8^{a} | 0.0 ± 0.0^{a} |
| | 240 | 70.1 ± 11.8^{a} | 0.9 ± 0.3^{b} | 495.3 ± 107.4^{b,c,d} | 1 265.6 ± 174.4^{a,b} | 50.6 ± 8.8^{a} | 0.0 ± 0.0^{a} |
| | 300 | 79.0 ± 11.2^{a} | 1.0 ± 0.2^{b} | 421.4 ± 106.3^{d} | 1 527.9 ± 106.7^{a,b} | 55.6 ± 7.5^{a} | 0.0 ± 0.0^{a} |
| | 360 | 77.0 ± 11.1^{a} | 1.2 ± 0.1^{b} | 321.5 ± 92.4^{d} | 1 654.7 ± 224.7^{b} | 50.9 ± 6.8^{a} | 0.0 ± 0.0^{a} |
| ED2MBUF | 0 | 42.7 ± 3.9^{a} | 0.0 ± 0.0^{a} | 601.8 ± 43.5^{a} | 0.0 ± 0.0^{a} | 30.6 ± 3.1^{a} | 0.0 ± 0.0^{a} |
| configuration | | | | | | | |
| | 30 | 41.8 ± 4.9^{a} | 0.6 ± 0.0^{a} | 553.2 ± 42.1^{a} | 32.8 ± 11.3^{a,c} | 29.8 ± 3.9^{a} | 0.0 ± 0.0^{a} |
| | 60 | 40.5 ± 3.6^{a} | 1.3 ± 0.2^{b} | 499.4 ± 17.0^{a,b} | 66.1 ± 22.1^{a,c} | 28.9 ± 2.8^{a} | 0.0 ± 0.0^{a} |
| | 120 | 37.9 ± 3.3^{a} | 2.0 ± 0.2^{c} | 414.7 ± 17.4^{b,c} | 122.2 ± 22.2^{a,c} | 27 ± 2.7^{a} | 0.0 ± 0.0^{a} |
| | 180 | 35.6 ± 2.1^{a} | 2.2 ± 0.3^{c} | 338.2 ± 37.1^{c} | 182.7 ± 50.7^{c} | 25.5 ± 1.9^{a} | 0.0 ± 0.0^{a} |
| ED2UF | 0 | 61.6 ± 16.4^{a} | 0.0 ± 0.0^{a} | 824.4 ± 196.2^{a} | 976.8 ± 132.7^{a} | 44.7 ± 12.5^{a} | 0.0 ± 0.0^{a} |
| configuration | | | | | | | |
| | 30 | 41.3 ± 7.0^{a} | 0.0 ± 0.0^{a} | 720.2 ± 97.1^{a} | 543.7 ± 241.8^{a,b} | 29.4 ± 5.5^{a} | 0.0 ± 0.0^{a} |
| | 60 | 32.9 ± 5.8^{a,b} | 0.0 ± 0.0^{a} | 699.9 ± 104.2^{a} | 414.5 ± 212.0^{b} | 23.1 ± 4.8^{a,b} | 0.0 ± 0.0^{a} |
| | 120 | 16.2 ± 3.3^{a,b} | 0.2 ± 0.4^{a} | 486.9 ± 55.1^{a} | 320.6 ± 94.2^{b} | 10.0 ± 2.4^{a,b} | 0.0 ± 0.0^{a} |
| | 180 | 21.6 ± 14.1^{b} | 0.9 ± 0.7^{a} | 625.4 ± 300.0^{a} | 260.0 ± 37.0^{b} | 12.2 ± 11.2^{b} | 0.0 ± 0.0^{a} |

| | | Sodium | | Phosphorus | |
|---|---|---|---|---|---|
| | Time | Cranberry | Recovery | Cranberry | Recovery |
| | (min) | juice | solution | juice | solution |
| ED2MB | 0 | 63.5 ± 6.4^{a} | 46.3 ± 32.7^{a} | 28.5 ± 3.5^{a} | 0.4 ± 0.0^{a} |
| configuration | | | | | |
| | 60 | 74.2 ± 14.0^{a} | 106.8 ± 60.3^{a} | 22.2 ± 1.1^{a,b} | 11.1 ± 1.4^{a,b} |
| | 120 | 69.7 ± 17.9^{a} | 101.6 ± 81.3^{a} | 13.5 ± 1.9^{c,d} | 15.5 ± 6.9^{b,c} |
| | 180 | 64.5 ± 17.7^{a} | 183.4 ± 106.5^{a} | 7.9 ± 1.2^{d,e} | 26.3 ± 3.8^{b,c,d} |
| | 240 | 55.7 ± 12.7^{a} | 178.4 ± 109.2^{a} | 5.1 ± 1.5^{e} | 24.4 ± 6.3^{c,d} |
| | 300 | 55.5 ± 17.2^{a} | 216.3 ± 100.2^{a} | 3.4 ± 1.2^{e} | 30.4 ± 2.1^{d} |
| | 360 | 48.2 ± 16.0^{a} | 248.5 ± 97.2^{a} | 2.4 ± 1.0^{e} | 32.9 ± 4.3^{d} |
| ED2MBUF | 0 | 37.0 ± 2.6^{a} | 15.1 ± 8.2^{a} | 20.8 ± 1.7^{a} | 0.4 ± 0.0^{a} |
| configuration | | | | | |
| | 30 | 57.6 ± 16.2^{a,b} | 50.9 ± 5.2^{b} | 20.5 ± 1.8^{a} | 0.4 ± 0.0^{a} |
| | 60 | 85.9 ± 30.3^{a,b} | 66.0 ± 0.9^{b,c} | 19.8 ± 0.9^{a} | 0.4 ± 0.0^{a} |
| | 120 | 136.4 ± 50.7^{a,b} | 88.6 ± 5.1^{c,d} | 19.6 ± 1.6^{a} | 0.4 ± 0.0^{a} |
| | 180 | 190.6 ± 68.9^{b} | 115.2 ± 18.1^{d} | 19 ± 1.3^{a} | 0.4 ± 0.0^{a} |
| ED2UF | 0 | 57.8 ± 15.5^{a} | 8.5 ± 2.1^{a} | 28.8 ± 7.7^{b} | 0.4 ± 0.0^{a} |
| configuration | | | | | |
| | 30 | 165.4 ± 57.0^{a} | 91.0 ± 37.4^{a} | 24.1 ± 4.7^{b} | 0.4 ± 0.0^{a} |
| | 60 | 262.1 ± 85.1^{a,b} | 164.1 ± 82.6^{b,c} | 24.4 ± 5.8^{b} | 0.4 ± 0.08^{a} |
| | 120 | 420.6 ± 103.5^{a,b} | 295.5 ± 78.9^{c,d} | 19.3 ± 1.1^{b} | 1.1 ± 0.6^{a} |
| | 180 | 620.4 ± 209.5^{b} | 426.6 ± 58.9^{d} | 27.2 ± 15.3^{b} | 1.9 ± 1.1^{a} |

*Data in the same column with different letters for the same parameter are significantly different between them at a probability level of 0.05.

### Juice Color:

Juice color, as measured using L*a*b* parameters remained unchanged during treatments under all tested configurations (P>0.05).

It is well known that four major pigments (cyanidin-3-galactoside, peonidin-3-galactoside, cyanidin-3-arabinoside and peonidin-3-arabinoside) are responsible for red color of cranberry juice²³. Because all three configurations showed no significant variation in L*a*b* parameters, it is likely that no significant interactions occurred between polyphenol and ED membrane or between polyphenols and anthocyanins.

### Organic Acid Content:

Major organic acids in cranberry juice are quinic acid (MW=192.17 g·mol⁻¹, pKa=3.46), citric acid (MW=192.12 g·mol⁻¹, pKa₁=3.13, pKa₂=4.76, pKa₃=6.39), malic acid (MW=134.09 g·mol⁻¹, pKa₁=3.46, pKa₂=5.05) and succinic acid (MW=118.09 g/mol, pKa₁=4.03, pKa₂=5.28)^{5,13,24}.

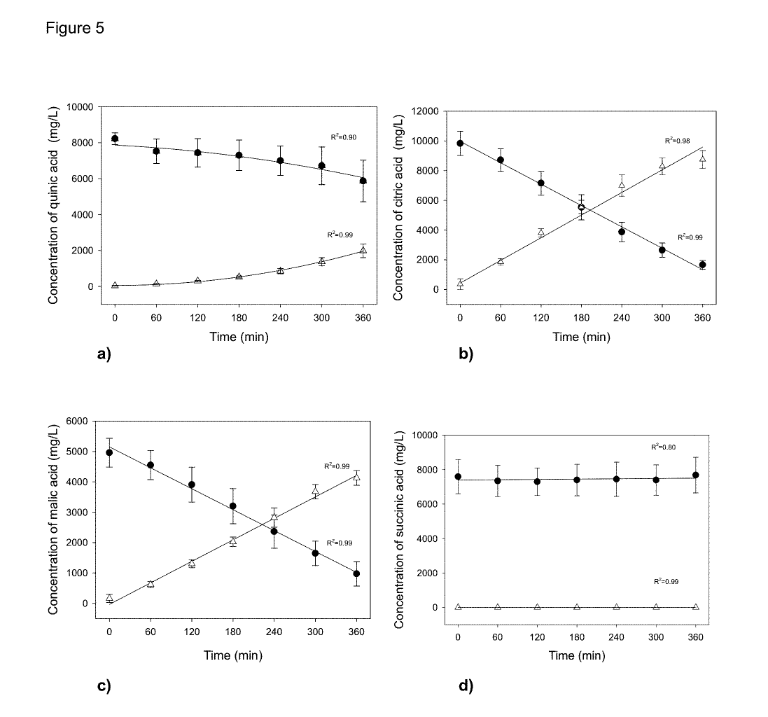

Based on the titratable acidity previous measurements, the concentrations of individual organic acid was only determined for the ED2 MB configuration (FIG. 5).

The concentration of succinic acid remained unaffected throughout the treatment (P>0.05). Indeed, succinic acid (pKa₁=4.03) is mainly in the non-dissociated and uncharged state at the juice pH, therefore no migration can occur through the anionic membrane. However, the ED2 MB cell configuration had a significant effect on the migration of the remaining organic acids including quinic, malic and citric acid. The level of citric acid decreased at a rate of 22.6 ppm/min in cranberry juice. After a three-hour treatment, the level of citric acid in the juice was reduced by 43.8%, whereas a reduction of 83% was reached after six hours. In contrast, the migration rate of malic acid (11.07 ppm/min) was twice as lower as citric acid. Concerning quinic acid, its migration rate was statistically unchanged in cranberry juice during the six hours of treatments but quinic concentration has a tendency to increase in the recovery solution: its seems that quinic acid would only migrate after two or three hours of treatment.

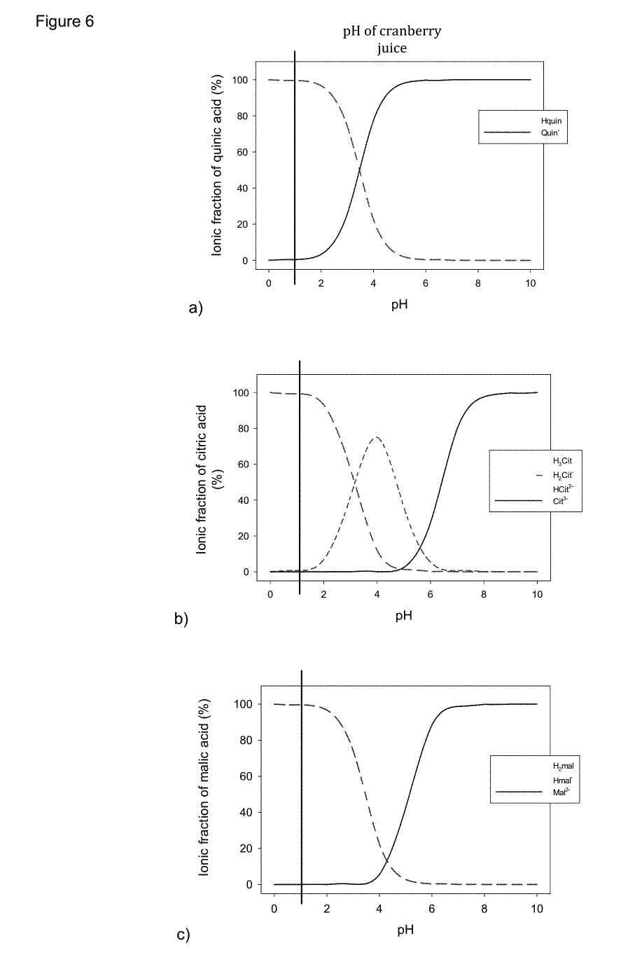

Due to its pKa value and its lower molecular weight, the migration of citric acid (the most abundant organic acid in cranberry juice) was earlier and relatively faster (22.6 ppm/min) than other organic acids. Hence, the migration rate of other acid such as malic acid was slower in the first half of the treatment (9.8 ppm/min) and then increased in the second half (12.4 ppm/min). The fast depletion of citric acid in cranberry juice may explain the accelerated migration further observed for other organic acids, caused by an increase in their concentration. The selective migration of organic acids adds value to the ED2 MB configuration because quinic acid is a specific organic acid and one of the components specific to cranberry juice and, citric and malic acids migrated significantly at different rate during the process. To our knowledge, such a selective migration of organic acid has never been reported in the literature. As mentioned before, preserving quinic acid in the treated juice is essential, as this acid is used for cranberry juice authentification¹¹²⁵. In cranberry juice, in addition to different pKa values, citric acid and malic acid have three or two negative charges whereas quinic acid has only one which may explain that they migrated differently through the anion-exchange membrane, and during the process (FIG. 6). The lower migration rate of quinic acid compared to citric and malic acid was previously observed in capillarity electrophoresis²⁶. Quinic acid is composed of an aromatic carboxycilic ring that has a high steric hindrance, which decreases its migration through anion-exchange membrane.

### Anthocyanin Content:

The major anthocyanin compounds in cranberry juice are cyanidin-3-galactoside, cyanidin-3-glucoside, cyanidin-3-arabinoside, peonidin-3-galactoside, peonidin-3-glucoside and peonidin-3-arabinoside. As for organic acids, the individual concentrations of these compounds were only measured for the ED2 MB configuration and are presented in Table 3. Results showed no significant variation in the level of individual anthocyanin in cranberry juice, which was further confirmed by the no detection of anthocyanin in the recovery compartment. Anthocyanins did not migrate through the anionic exchange membrane because these molecules are positively charged at the pH of cranberry juice which is associated with a low diffusion coefficient²⁷. Consequently, the quality of cranberry juice in terms of anthocyanin content was preserved with ED2 MB configuration.

Proanthocyanidin content:

Statistical analysis of proanthocyanidins (PACs) data revealed that ED2 MB configuration had no significant effect on total proanthocyanidin concentrations (Table 3). The average total proanthocyanidins of cranberry juice was 106.8±5.1 ppm and remained more or less constant (P>0.05). Proanthocyanidins were not detected in the recovery solution. As anthocyanins compounds, proanthocyanidins are positively charged at the pH value of cranberry juice thus do not migrate through anionic exchange membrane²⁷.

TABLE 3
Concentrations of individual anthyocanins (mg/L), proanthocyanidins (mg/L) and
total polyphenols content (mg/L gallic acid equivalent) in cranberry juices treated
using the ED2MB configuration, and in control juice.

| Time | Anthocyanins | | | | | |
|---|---|---|---|---|---|---|
| (min) | Cyd-3-gal | Cyd-3-glu | Cyd-3-arab | Pnd-3-gal | Pnd-3-glu | Pnd-3-arab |
| 0 | 13.8 ± 1.2^{a} | 0.3 ± 0.0^{a} | 14.4 ± 1.3^{a} | 20.0 ± 1.9^{a} | 1.8 ± 0.2^{a} | 10.8 ± 1.0^{a} |
| 60 | 13.2 ± 1.4^{a} | 0.3 ± 0.0^{a} | 13.7 ± 1.5^{a} | 19.1 ± 2.1^{a} | 1.7 ± 0.2^{a} | 10.3 ± 1.1^{a} |
| 120 | 13.1 ± 1.4^{a} | 0.3 ± 0.0^{a} | 13.6 ± 1.5^{a} | 19.1 ± 2.1^{a} | 1.7 ± 0.2^{a} | 10.3 ± 1.1^{a} |
| 180 | 13.1 ± 1.3^{a} | 0.2 ± 0.0^{a} | 13.5 ± 1.5^{a} | 19.0 ± 2.1^{a} | 1.7 ± 0.2^{a} | 10.2 ± 1.1^{a} |
| 240 | 12.9 ± 1.3^{a} | 0.3 ± 0.0^{a} | 13.4 ± 1.3^{a} | 18.9 ± 1.9^{a} | 1.7 ± 0.2^{a} | 10.2 ± 1.1^{a} |
| 300 | 12.8 ± 1.3^{a} | 0.3 ± 0.0^{a} | 13.2 ± 1.3^{a} | 18.8 ± 1.8^{a} | 1.7 ± 0.2^{a} | 10.1 ± 1.0^{a} |
| 360 | 13.0 ± 1.0^{a} | 0.2 ± 0.0^{a} | 12.9 ± 1.3^{a} | 18.6 ± 1.9^{a} | 1.7 ± 0.2^{a} | 10.0 ± 1.0^{a} |
| | Proanthocyanidins | | | | | |
| | Monomers | 2-3 mers | 4-6 mers | 7-10 mers | Polymers | Totals |
| 0 | 19.7 ± 1.9^{a} | 23.4 ± 9.4^{a} | 6.5 ± 6.0^{a} | 0.0 ± 0.0^{a} | 30.4 ± 2.5^{a} | 106.6 ± 11.2^{a} |
| 60 | 18.0 ± 2.1^{a} | 21.1 ± 10.0^{a} | 5.5 ± 4.5^{a} | 0.3 ± 0.7^{a} | 28.4 ± 4.1^{a} | 97.8 ± 16.0^{a} |
| 120 | 19.3 ± 1.8^{a} | 21.1 ± 9.8^{a} | 5.4 ± 4.5^{a} | 0.4 ± 1.2^{a} | 34.9 ± 5.2^{a} | 99.2 ± 11.6^{a} |
| 180 | 18.2 ± 2.8^{a} | 21.2 ± 9.7^{a} | 5.5 ± 4.6^{a} | 0.1 ± 0.2^{a} | 40.5 ± 4.8^{a} | 110.9 ± 17.1^{a} |
| 240 | 18.6 ± 2.3^{a} | 21.0 ± 9.7^{a} | 5.5 ± 4.5^{a} | 0.0 ± 0.0^{a} | 41.3 ± 5.5^{a} | 111.2 ± 12.9^{a} |
| 300 | 18.8 ± 1.9^{a} | 20.8 ± 9.7^{a} | 5.4 ± 4.4^{a} | 0.0 ± 0.0^{a} | 41.4 ± 6.8^{a} | 110.8 ± 14.0^{a} |
| 360 | 18.2 ± 1.9^{a} | 20.3 ± 9.5^{a} | 5.3 ± 4.4^{a} | 0.0 ± 0.0^{a} | 43.1 ± 9.5^{a} | 110.9 ± 17.1^{a} |

Total Polyphenols

| Time (min) | Polyphenols |
|---|---|
| 0 | 1031 ± 94^{a} |
| 60 | 1091 ± 44^{a} |
| 120 | 1134 ± 117^{a} |
| 180 | 1128 ± 179^{a} |
| 240 | 1101 ± 132^{a} |
| 300 | 1052 ± 142^{a} |
| 360 | 1003 ± 229^{a} |

*Data in the same column with different letters for the same parameter are significantly different at a probability level of 0.05.

### Total Polyphenols:

There was no significant difference in total polyphenols content (P>0.05, Table 3). The total averaged polyphenols content of cranberry juice was found to be constant all over the process at a level of 1074±136 mg/L gallic acid equivalents comparable to values reported¹⁷: no migration of polyphenols was observed. Therefore, it could also be concluded that no interactions between polyphenols and membrane were observed.

### Electrodialytic Parameters:

### Global System Resistance:

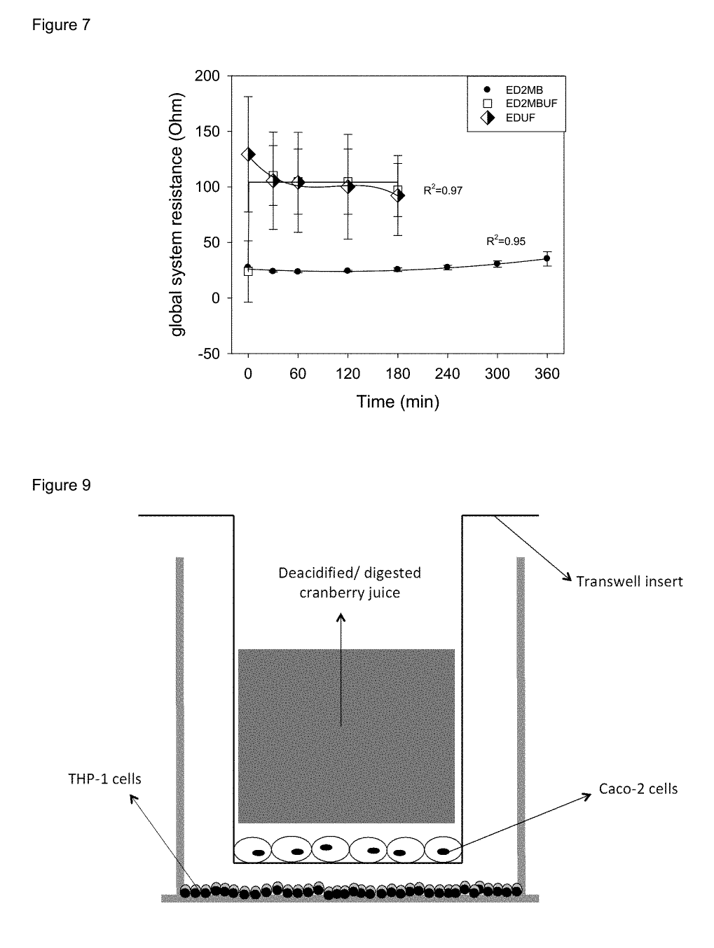

According to the analysis of variance, in ED2 MB configuration, the global system resistance increased significantly from 27.5±0.4 to 35.2±6.4Ω (P<0.001) whereas this variable remained constant in the ED2MBUF and EDUF configuration (FIG. 7). The global system resistance was higher in systems involving UF membrane because they are generally less conductive than ion exchange membranes.

### Membranes Conductivities and Thicknesses:

Based on t-tests, every configuration tested showed no significant changes either in membrane conductivity or membrane thickness, before and after the ED treatment (data not shown). These results are in agreement with those obtained for the global system resistance; consequently, it can be concluded that the fouling of membranes was not significant whatever the configuration. In addition, only ion-exchange membranes in the ED2 MB configuration were not colored by the cranberry juice compounds (polyphenol compounds) after eighteen hours of treatments (three replications of six hours).

### CONCLUSION

The aim of the present study was to deacidify cranberry juice using an electrochemical process named electrodialysis. Three different configurations using different combinations of ion-exchange and/or ultrafiltration membranes were tested in order to determine the most efficient and to provide an optimal deacidification level without significantly decreasing the quinic acid content. However, the treated volume solution, the membrane type and the total membrane specific area used would influence the deacidification time. In terms of migration rates and physicochemical parameters (juice color, anthocyanin, proanthocyanidin, organic acid and ion concentration contents), it appeared from these results that the ED2 MB configuration was the most effective one. With this configuration, a deacidification level of 40% was obtained in cranberry juice, after three hours of treatment. In the same time, purified organic acids, or mixed organic acids were recovered in the recovery solution; these purified organic acids can be used as preservative and/or flavoring agents, in various food applications²⁸.

Compared to the ED2 MB configuration, the principal limitation of the ED2MBUF configuration appeared to be the introduction of an ultrafiltration membrane, negatively charged at cranberry juice pH, and consequently slowing down or stopping migration. It would be interesting to test a positively charged ultrafiltration membrane and see if similar results are obtained on the evolution of physicochemical parameters. Furthermore, to compare the different cell configurations, it appears that the contribution of negatively charged molecules or OH⁻ molecules, in cranberry juice, is necessary to the migration of organic acids. To confirm this hypothesis, it would be interesting to study the presence of negatively charged molecules such as OH— ions and their impact on the dissociation kinetic of organic acid in juice, and their migration to the recovery compartment with model solution.

Based on the results obtained in this study, ED2 MB configuration was chosen for scaling-up cranberry juice deacidification and to bring a new product to the market.

### Example 2—Deacidification of Cranberry Juice Protects Against Disruption of In-Vitro Intestinal Cell Barrier Integrity

### Materials and Methods

### Cranberry Juice

Treatments were carried out on a pasteurized and clarified cranberry juice produced by Fruit d'Or (Notre-Dame-de-Lourdes, QC, Canada). This raw juice was diluted to obtain a value of 8° Brix, then stored at −20° C. and thawed at 4° C. before each experiment. The physicochemical characteristics of the cranberry juice used in this experiment are presented in Table 4.

TABLE 4
Physicochemical characteristics of the raw cranberry juice.

| | |
|---|---|
| pH | 2.35 ± 0.03 |
| Conductivity (mS/cm) | 2.7 ± 0.5 |
| Titratable acidity (g/L of citric | 9.1 ± 0.6 |
| acid monohydrate equivalents) | |
| Total soluble solids (° Brix) | 7.2 ± 0.5 |
| Total proanthocyanidins (mg/L) | 210.7 ± 7.3 |
| Total polyphenols (mg/L of gallic | 1 001 ± 101 |
| acid equivalents) | |
| Anthocyanins (mg/L) | |
| Cyanidin-3-galactose | 38.2 ± 0.2 |
| Cyanidin-3-glucose | 1.2 ± 0.0 |
| Cyanidin-3-arabinose | 37.9 ± 0.2 |
| Peonidin-3-galactose | 53.7 ± 0.3 |
| Peonidin-3-glucose | 3.8 ± 0.0 |
| Peonidin-3-arabinose | 28.6 ± 0.2 |
| Organic acids (mg/L) | |
| Quinic acid | 12 167 ± 1 676 |
| Citric acid | 21 451 ± 2 383 |
| Malic acid | 13 794 ± 1 837 |
| Succinic acid | 3 021 ± 686 |

### Deacidification Treatment

The cell configuration and the electrodialytic protocol were similar to those used in Example 1 and by Serre et al. (2016) but at a pilot scale. Cranberry juice with different final deacidification levels was produced on a pilot scale after 0, 20, 40, 60 and 80 min of treatment.

### In Vitro Digestion

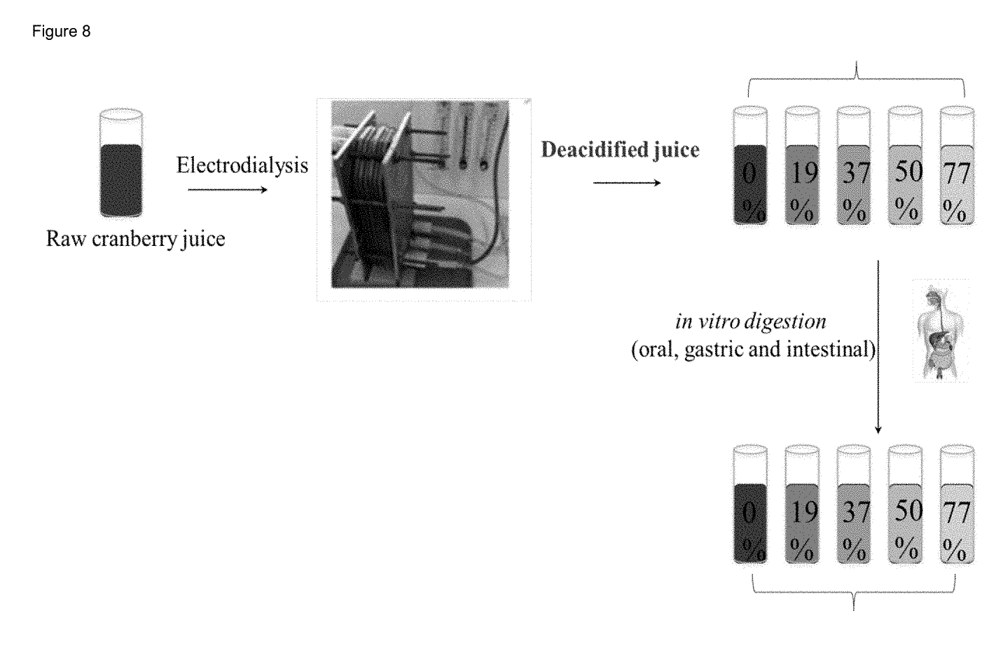

According to Versantvoort et al. (2005), an in vitro digestion process was simulated on treated and untreated cranberry juice (FIG. 8). Briefly, samples (12 g) were mixed with simulated saliva (6 mL at pH 6.8±0.2) and incubated at 37° C. for 5 min without agitation. Gastric juice (6 mL at pH 1.3±0.1) was added and each sample was rotated for 2 h at 37° C. During this period, the pH of each sample was adjusted to 2.4±0.1 with 5 M HCl. Finally, intestinal juice (12 mL at pH 8.1±0.2), bile juice (6 mL at pH 8.2±0.2) and bicarbonate solution (2 mL) were added and agitated for 2 h at 37° C. In duodenal juice, lipase was not added since it is already present in porcine pancreatin (Sigma-Aldrich, Saint Louis, Mo., USA). Three replicates of each sample were in vitro digested, frozen at −20° C. and thawed at 4° C. before analyses of organic acid, proanthocyanidin, anthocyanin, total polyphenol content and oxygen radical absorbance capacity. The in vitro digested cranberry juice was centrifuged at 3250×g for 15 min and filtered using a 0.22 μm filter prior to use.

### Co-Culture System

The Caco-2 cells and the human leukemia monocytic cell line (THP-1) were obtained from the American Type Culture Collection (ATCC, Manassas, Va., USA). Caco-2 cells were seeded at 3×10⁵ cells/well onto Transwell insert plates (12 mm diameter, 0.4 μm pore size; Costar Corp., Cambridge, Mass., USA) and grown in Dulbecco's Modified Eagle's Medium (DMEM) supplemented with 10% fetal bovine serum, 1% non-essential amino acid solution, 1 mM sodium pyruvate, 100 μg/mL streptomycin, and 100 U/mL penicillin (all from Hyclone, Logan, Utah, USA) at 37° C., 5% CO₂. The media was changed every 3 days until the cells were fully differentiated (21 days). Cells were used at passage numbers 32-41. On day 21, the integrity of the cell monolayer was confirmed by transepithelial electrical resistance (TEER) using Millicell®-ERS (Millipore, Bedford, Mass., USA). Confluent cell monolayers produced TEER values greater than 1000 Ω·cm² after correction for resistance in blank control wells.

THP-1 cells were grown in RPMI supplemented with 10% fetal bovine serum, 100 μg/mL streptomycin, and 100 U/mL penicillin (all from Hyclone, Logan, Utah, USA)). The cells were plated at a density of 5×10⁵ cells/well in 6-well plates and incubated for 72 h in the presence of 100 nM phorbol myristate acetate (PMA from Sigma, St. Louis, Mo., USA) to differentiate them into a macrophage-like phenotype.

After replacing all media with Hank's Balanced Salt Solution (HBSS from GE, Logan, Utah, USA), the Transwell inserts with cultured Caco-2 cells were added to multiple plate wells preloaded with THP-1 cells (see FIG. 9). The HBSS was then removed from the apical compartment and replaced with 500 μL of in vitro digested cranberry juice. HBSS or a solution of 1% Triton in HBSS were used as negative and positive controls, respectively. The inserts were incubated for 3 h at 37° C., 5% CO₂. The TEER was measured before and after adding cranberry juice. Results were expressed as the change in TEER measurement (ΔTEER).

### Analyses

### Titratable Acidity

The titratable acidity of both recovery solution and cranberry juice was measured using AOAC method no. 942.15 (2005) during and after deacidification treatment. These solutions (4 mL) were titrated with 0.1 M NaOH to pH 8.2. The titratable acidity (TA) was expressed as g/L of citric acid monohydrate equivalents.

### Organic Acid Content

The content of the three main organic acids (quinic, malic and citric) was measured in both recovery solution and cranberry juice, and extracted using C18-SPE cartridges (non endcapped 6 mL, 500 mg, Silicycle, Quebec city, QC, Canada). The cartridges were conditioned with methanol (5 mL), washed with distilled water (5 mL), and acetonitrile/water (50% (v/v), 10 mL). After the cartridges were dried, samples (10 mL) were taken, and 5 mL was used for HPLC analysis.

Following AOAC method no. 986.13 (2000), a Waters HPLC system (Milford, Mass., USA) equipped with a UV detector (Waters, model 966) was used to analyse the individual organic acid content. Briefly, organic acids were separated on a Synergi Hydro-RP column (250*4 mm, Phenomenex, Torrance, Calif., USA) using a mobile phase composed of a 0.2 M (v/v) KH₂PO₄, at a flow rate of 0.8 mL/min. The detection wavelength was 214 nm. Based on the calibration curves of authentic standards (Sigma Company, Saint Louis, Mo., USA) and their retention times, citric, malic and quinic acids were identified and quantified.

### Anthocyanin Content

Individual anthocyanin content was analyzed by HPLC (Wrolstad, 2004). Briefly, an Agilent 1100 series (Agilent technologies, Santa Clara, Calif., USA) equipped with a diode array detector was used. Samples (0.5 mL) were injected and analyzed with a Luna 5 μm C₁₈ column (2*250 mm, Phenomenex, Torrance, Calif., USA). Two solvents were used for elution at 1 mL/min.: solvent A, 100% acetonitrile and solvent B, acetic acid/acetonitrile/phosphoric acid (10:5:1) in water. The detection wavelength was 520 nm. Using a molar extinction coefficient of 26,900 and a molecular weight of 4,492 g/mol, individual anthocyanin composition could be quantified in mg/L of cyanidin-3-glucose equivalents.

### Proanthocyanidin Content

Individual proanthocyanidin content was determined using an Agilent 1100 series HPLC system equipped with a fluorescence detector (Waters, model 474, Milford, Calif., USA) (Khanal, Howard, Brownmiller, & Prior, 2009). Briefly, proanthocyanidins (PACs) were analyzed with a Luna 5 μm silica column (3*150 mm, Phenomenex, Torrance, Calif., USA) according to their degree of polymerization, using a linear gradient from 0% to 40% B, in 35 min.; 40% to 100% B, in 40 min.; 100% isocratic B, in 45 min.; and 100% to 0% B, in 50 min. Two solvents were used at a flow rate of 0.65 solvent A, dichloromethane/methanol/acetic acid/water (82:14:2:2) and solvent B, methanol/acetic acid/water (96:2:2). The emission and excitation wavelengths were 321 nm and 230 nm, respectively.

For each polymerization degree, the epicatechin curve was modified with a relative equivalent factor. Individual proanthocyanidin content was expressed as mg/litre of epicatechin equivalents.

### Total Polyphenols

Waterhouse's microscale protocol for Folin-Ciocalteau colorimetry (Waterhouse, 2002) was used to determine total polyphenols. The system was equipped with a UV-visible spectrometer (Agilent Technologies, Palo Alto, Calif., USA) using a detection wavelength at 765 nm. Total polyphenol concentrations were expressed in gallic acid equivalents per liter of juice (mg/L).

### Oxygen Radical Absorbance Capacity (ORAC)

The oxygen radical absorbance capacity assays were carried out with a Galaxy fluorometer (BGM LabTech, Durham, N.C., USA) (Cao, Alessio, & Cutler, 1993; Wada & Ou, 2002). Briefly, samples were diluted (1/900 for deacidified cranberry juice and 1/300 for deacidified and digested cranberry juice) in 0.075 M phosphate buffer (pH 7.0). Then, 200 μL of a fluorescein solution (0.036 mg/mL), 20 μL of diluted sample and 75 μL of 2,2′-azobis-2-aminopropane dihydrochloride (AAPH) (8.6 mg/mL) were added to each well. The emission and excitation wavelengths were 520 nm and 485 nm, respectively, and the fluorescence was recorded 35 times during the 120 min analysis. The ORAC results were calculated on the basis of a Trolox standard curve (a water-soluble analog of vitamin E) and reported as micromoles of Trolox equivalent (TE) per liter of treated juice.

### Statistical Analyses

SAS software (version 6.3 for windows, SAS Institute Inc., Cary, N.C., USA) and GraphPad Prism (version 6.05 for Windows, GraphPad Software, La Jolla, Calif., USA) were used to perform analysis of variance (ANOVA), Student tests, Tukey tests and Dunnett tests. Significant differences were declared at probability level P<0.05.

### Results

### Deacidification of Cranberry Juice

To calculate the deacidification level, the following equation was used:

deacidification level(in %)=(1−TA₍ₜ₎/TA₍ₜ₌₀₎)*100 (1)

where TA is the titratable acidity. Cranberry juice samples with different final deacidification rates were produced at a pilot scale after 0, 20, 40, 60 and 80 minutes of treatment corresponding to 0, 19, 37, 50 and 77% deacidification, respectively. The deacidification level was linear as a function of the deacidification process duration (R²=0.98), as demonstrated by the following equation:

Y=0.9317*X (2)

where Y is the deacidification level (in %) and X is the duration of the deacidification process (in min.).

This result confirmed previous results obtained in Example 1 and by Serre et al. (2016) on a laboratory scale.

### Physico-Chemical Characteristics of Deacidified and Deacidified/Digested Cranberry Juices

### Organic Acid Content

During electrodialysis (ED) treatment of the cranberry juice, citric and malic acids decreased significantly. After 80 min of treatment, 74% of citric acid and 93% of malic acid had migrated into the recovery solution. Quinic acid remained constant until 50% deacidification (P=0.0780). The concentrations of organic acids remained constant between deacidified cranberry juice and deacidified/digested cranberry juice (P between 0.06 and 0.54 as a function of treatment time; Table 5). As expected, the concentration of organic acids changed according to their chemical structures and pKas (migration of 89.8, 76.6 and 36.6% after 80 min of treatment for malic, citric and quinic acid, respectively), following the duration of the deacidification process (quinic acid migrated only after 60 min of treatment), as already reported by Serre et al. (2016). Indeed, these three major organic acids have different molecular weights, chemical structures, and numbers and values for pKa: quinic acid (MW=192.17 g·mol-1, pKa=3.46, possesses an aromatic carboxycyclic ring as part of its structure), citric acid (MW=192.12 g·mol-1, pKa1=3.13, pKa2=4.76, pKa3=6.39) and malic acid (MW=134.09 g·mol-1, pKa1=3.46, pKa2=5.05) (Husson et al., 2013; Marietta, 1985; Wing et al., 2008). Consequently, due to its pKa values and its lower molecular weight, the migration of citric acid (the most abundant organic acid in cranberry juice) was earlier and faster than malic and quinic acids (Serre et al., 2016). In addition quinic acid is composed of an aromatic carboxycyclic ring with high steric hindrance, which decreases its migration through an anion-exchange membrane (Serre et al., 2016).

The digestion process did not significantly affect the organic acid content in cranberry juice.

### Proanthocyanidins

Significant differences in PAC content were observed in digested deacidified cranberry juice compared to deacidified cranberry juice (Table 6). The 7-10 degree of polymerization (DP; P<0.05) and 4-6 DP (P<0.05) groups of PACs disappeared after digestion. Polymers of DP>10 (P<0.05) and monomers (P<0.05) decreased from 34±9 to 16±2 ppm and from 26±4 to 4±1 ppm, respectively, after digestion. For deacidified cranberry juice, the digestion process increased the 2-3 DP of PACs from 109±16 to 184±19 ppm (P<0.05). The average total PAC content was close to 213±15 ppm and did not change during the digestion process although some differences appeared at 0% and 19% (P=0.008 for 0%; P=0.020 for 19%; P=0.057 for 37%; P=0.125 for 50% and P=0.135 for 77%). However, these differences are very low compared to other data and could be due to the sensitivity of the analytical method.

### Anthocyanins

Individual anthocyanin concentrations were not detected after the digestion process, in contrast to deacidified cranberry juice (data not shown, since all data after digestion are equal to 0 ppm). The digestion process appears to degrade anthocyanins present in cranberry juice. Indeed, it has been demonstrated that anthocyanins are sensitive to alkaline conditions and digestive enzymes (Bermudez-Soto, Tomasbarberan, & Garciaconesa, 2007).

### Total Polyphenols

In deacidified cranberry juice, the total polyphenol concentration remained constant for all deacidification conditions (P=0.3298).

The digestion of deacidified cranberry juice had a significant impact (P<0.05) (Table 7). During in vitro digestion, the total polyphenol concentration decreased from 927±125 to 289±9 ppm, corresponding to a 3-fold decrease. This result is due to the degradation of PACs of lower DP or monomers, and to the sensitivity of polyphenols, such as catechins (monomers of PACs) (Krueger et al., 2016) and anthocyanins, to alkaline pH conditions and the presence of pancreatin during digestion (Bermudez-Soto et al., 2007; Kumamoto, Sonda, Nagayama, & Tabata, 2001; Sharma & Zhou, 2011).

### Antioxidant Capacity

During ED treatment, antioxidant capacity remained quite constant, taking into account the large standard deviations observed. For deacidified cranberry juice, the digestion process had a significant impact: the antioxidant capacity decreased from 19795±2065 to 7733±1631 TE/L of juice, corresponding to a 2.5-fold decrease (Table 8). The decrease in the antioxidant capacity of digested treated cranberry juice correlated to the decrease in total polyphenol concentration as well as to the intestinal pH (7.0). Total polyphenol compounds largely contribute to the antioxidant capacity (Chen et al., 2014; Plumb, De Pascual-Teresa, Santos-Buelga, Cheynier, & Williamson, 1998).

### Evolution of Inflammatory Potential in Caco-2 Cells

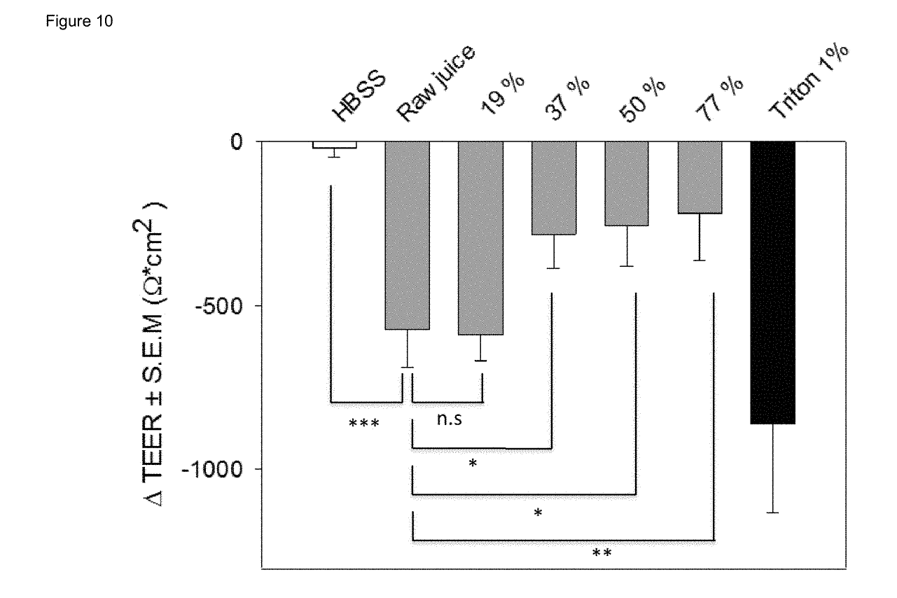

The impact of deacidified cranberry juice on the epithelial cell barrier was determined by measuring TEER (FIG. 10). Results are shown as the change in TEER values (ΔTEER). Negative TEER means that the barrier integrity is compromised. HBSS and 1% Triton were used as controls. Incubation with HBSS for 3 hours did not affect the TEER. The TEER was −20±28 Ω·cm². However, 1% Triton considerably modified cell integrity with a mean TEER value of −863±271 Ω·cm².

TABLE 6
Evolution of proanthocyanidins (mg/L) in deacidified and digested cranberry juice
relative to deacidified cranberry juice.

| | Monomer | | 2-3 mers | | 4-6 mers | |
|---|---|---|---|---|---|---|
| | Deacidified | | Deacidified | | Deacidified | |
| | cranberry juice | | cranberry juice | | cranberry juice | |
| Deacidification | Non | | Non | | Non | |
| level (%) | digested | Digested | digested | Digested | digested | Digested |
| 0 | 27.6 ± 0.5^{a}* | 5.0 ± 0.9^{b} | 113.7 ± 1.4^{a} | 202.4 ± 10.0^{b} | 37.0 ± 0.4^{a} | 12.5 ± 11.2^{b} |
| 19 | 23.6 ± 0.0^{a} | 3.7 ± 0.7^{b} | 98.6 ± 0.5^{a} | 196.9 ± 1.6^{b} | 33.0 ± 0.3^{a} | 0.5 ± 0.8^{b} |
| 37 | 22.5 ± 0.4^{a} | 3.7 ± 0.6^{b} | 94.0 ± 0.3^{a} | 188.4 ± 6.2^{b} | 31.7 ± 0.2^{a} | 0 ± 0^{b} |
| 50 | 28.5 ± 0.0^{a} | 5.3 ± 0.2^{b} | 119.6 ± 0.2^{a} | 186.5 ± 3.8^{b} | 40.8 ± 0.3^{a} | 0 ± 0^{b} |
| 77 | 22.8 ± 0.2^{a} | 3.1 ± 0.2^{b} | 93.6 ± 2.3^{a} | 177.1 ± 1.8^{b} | 32.2 ± 0.6^{a} | 0 ± 0^{b} |

| | 7-10 mers | | Polymers | | Total | |
|---|---|---|---|---|---|---|
| | Deacidified | | Deacidified | | Deacidified | |
| | cranberry juice | | cranberry juice | | cranberry juice | |
| Deacidification | Non | | Non | | No | |
| level (%) | digested | Digested | digested | Digested | digested | Digested |
| 0 | 4.7 ± 0.2^{a} | 0 ± 0^{b} | 27.7 ± 6.7^{a} | 17.9 ± 1.0^{b} | 210.7 ± 7.3^{a} | 237.9 ± 4.2^{b} |
| 19 | 3.9 ± 0.5^{a} | 0 ± 0^{b} | 30.6 ± 0.11^{a} | 17.6 ± 2.4^{b} | 186.3 ± 6.1^{a} | 218.7 ± 4.2^{b} |
| 37 | 4.0 ± 0.4^{a} | 0 ± 0^{b} | 32.5 ± 1.8^{a} | 16.3 ± 3.3^{b} | 178.9 ± 6.3^{a} | 208.4 ± 9.7^{a} |
| 50 | 5.2 ± 0.3^{a} | 0 ± 0^{b} | 35.6 ± 0.4^{a} | 14.6 ± 2.3^{b} | 224.9 ± 7.1^{a} | 206.4 ± 4.9^{a} |
| 77 | 3.6 ± 0.0^{a} | 0 ± 0^{b} | 38.4 ± 0.2^{a} | 15.1 ± 0.6^{b} | 189.8 ± 2.2^{a} | 195.3 ± 1.6^{a} |

*Data on the same raw with different letters for the same group of PACs are significantly different at a probability level of 0.05.

TABLE 7
Evolution of total polyphenol concentrations (mg/L of
gallic acid equivalents) in deacidified cranberry juice,
and in deacidified/digested cranberry juice.

Deacidified cranberry juice

| Deacidification level (%) | Non digested | Digested |
|---|---|---|
| 0 | 1001 ± 101^{a*} | 325 ± 30^{b} |
| 19 | 978 ± 73 ^{a} | 273 ± 16 ^{b} |
| 37 | 745 ± 12 ^{a} | 271 ± 8 ^{b} |
| 50 | 874 ± 55 ^{a} | 288 ± 25 ^{b} |
| 77 | 776 ± 67 ^{a} | 272 ± 4 ^{b} |

*Data on the same raw with different letters for the same parameter are significantly different at a probability level of 0.05

TABLE 8
Antioxidant capacity (in μM Trolox equivalent/L of juice samples)
in deacidified, and in deacidified/digested cranberry juice.

Deacidified cranberry juice

| Deacidification level (%) | Non digested | Digested |
|---|---|---|
| 0 | 20687 ± 998^{a*} | 6811 ± 1116^{b} |
| 19 | 19282 ± 88^{a} | 7831 ± 777^{b} |
| 37 | 19203 ± 473^{a} | 7592 ± 1132^{b} |
| 50 | 22690 ± 481^{a} | 10219 ± 1050^{b} |
| 77 | 17111 ± 1705^{a} | 6214 ± 574^{b} |

*Data on the same raw with different letters for the same parameter are significantly different at a probability level of 0.05

Each sample of deacidified/digested cranberry juice was compared to a digested raw cranberry juice. The deacidification level of 19% presented a similar ΔTEER value as the raw juice (P>0.05). However, for the other deacidification rates (37, 50 and 77%) the ΔTEER values increased significantly from −574±117 Ω·cm² to −253±22 Ω·cm², a 56% increase meaning increased protection of monolayer integrity.

Deacidified cranberry juice with a deacidification level of 37% or more did not decrease the integrity of Caco-2 cell monolayers. In contrast, raw cranberry juice and 19% deacidified juice produced a large decrease in the integrity of the monolayer. It appears from these results that at least 20% deacidification of the juice is required to protect the Caco-2 cell monolayer against a decrease in its integrity since 19% deacidification is not sufficient to protect the integrity of Caco-2 cells.

### DISCUSSION

During digestion of cranberry juice, the concentrations of the three main organic acids measured (citric, malic and quinic acids) remained unchanged during the three steps of the in vitro digestion, according to the initial deacidification level. The concentration of organic acids was only affected by the deacidification treatment duration as already demonstrated by Example 1 and Serre et al. (2016). Anthocyanins disappeared completely after digestion under all conditions tested. Indeed, anthocyanins are extremely sensitive to temperature, oxidation, enzymatic activity and pH, and their bioavailability is considered to be low (McGhie & Walton, 2007). Recent studies have demonstrated that anthocyanins are very sensitive to external factors such as alkaline pH and digestive enzymes. These compounds were analysed after intestinal digestion in the presence of digestive enzymes (the pH of all samples was approximately 7.0). All of these factors could explain the absence of anthocyanins in digested and deacidified cranberry juice (Fernandes, Faria, Calhau, de Freitas, & Mateus, 2014; Ou & Gu, 2014).

In contrast to anthocyanins, PACs are stable at alkaline pH (Ou & Gu, 2014). Some PACs disappeared while others increased during in vitro digestion for all deacidification rates. Hence, the amount of 2-3 DP doubled while other oligomers, notably 4-6 DP, 7-10 DP and polymers, decreased during the digestion process. This is confirmed by the recent study by Spencer et al. (2000) which demonstrated the depolymerization of decamers to trimers into dimers in a simulated gastric fluid (pH 2.0 at 37° C.). According to Ou and Gu (2014), PACs are absorbable as monomers or oligomers (DP<4) to a rate of 10% of epicatechin whereas the other polymers (DP>4) are non-absorbable in their intact form. Proanthocyanidins with a DP>4 are not absorbed due to their large molecular size and the gut barrier. Also, the bioavailability of these compounds decreases significantly with the DP (molecular size; Ou and Gu, 2014). Indeed, PACs, known as condensed tannins, are absorbed in the small intestine through passive diffusion across monolayers of human intestinal epithelial Caco-2 cells and form conjugated metabolites (glucuronidated, sulfated and/or methylated) in the liver. These metabolites are present in blood and tissues (Deprez, Mila, Huneau, Tome, & Scalbert, 2001; Romanov-Michailidis et al., 2012). The majority of these metabolites are degraded by gut microflora into phenolic acids and phenylvalerolactones (Gu, House, Rooney, & Prior, 2007; Sánchez-Patán et al., 2012). The absorption and metabolism of PACs are essential to human health. In fact, antioxidant activity due to phenolic compounds has the potential to reduce or delay the development of inflammation (Romier-Crouzet et al., 2009; Ruiz & Haller, 2006; Sergent, Piront, Meurice, Toussaint, & Schneider, 2010).

Of the different deacidification rates obtained, three (37, 50 and 77%) significantly protected the integrity of the Caco-2 cell monolayers. With a deacidification level over 37%, the integrity of CaCo-2 cell monolayers was only half as affected in comparison with the control raw juice. Indeed, the raw and the 19% deacidified cranberry juice had higher paracellular permeability of the monolayer, in comparison with deacidified juice at 37% and higher, demonstrating the impact of organic acid concentration on the integrity of the monolayer. Furthermore, our results suggest that quinic acid is not responsible for the loss of Caco-2 cell monolayer integrity, since its concentration was still constant up to 50% deacidification in raw and deacidified juices, while the TEER decreased beginning at 37% deacidification. There was no difference in TEER over the 37% deacidification level. Loss of integrity would be only due to malic and citric acid concentrations. In addition, the low pH of the cranberry juice did not affect monolayer integrity. The pH would be neutralized by gastric salts during digestion and would not further affect the intestinal cells. Organic acids have been reported to be involved in potential inflammation in the gastrointestinal tract (Steen et al., 1995; Welling et al., 2014) and when present at high concentrations, as in the raw cranberry juice, could lead to intestinal inflammation when cranberry juice is consumed over a long period. Such a side effect has been reported in clinical trials involving cranberry juice: 40% of withdrawn subjects report undesirable side effects (gastrointestinal troubles) (McMurdo et al., 2005; Wing et al., 2008). Such side effects have not been reported for clinical trials involving cranberry extract, since no or very low concentrations of organic acids are present in these extracts due to the process of extraction (Jepson, Williams, & Craig, 2012). Organic acids can impact cell monolayer integrity, as shown by studies demonstrating that exposure to citric add, which acts as a calcium chelator, can cause an increase in paracellular absorption by disrupting tight junction complexes, via depletion of intracellular calcium (Froment, Molitoris, Buddington, Miller; & Alfrey, 1989; Grant & Leone-Bay, 2012; Nolan, Califano, & Butzin, 1990). Calcium is essential for establishing epithelial cell junction networks in the intestine and for the integrity and sealing of tight junctions (Noach, Kurosaki, Blom-Roosemalen, de Boer; & Breimer, 1993; Tomita, Hayashi; & Awazu, 1996). According to Daugherty and Mrsny (1999), organic acids can affect the paracellular permeability (disruption of tight junction complex) as determined by a modification of TEER. Tight junction complexes are the primary barrier of paracellular transport. Each epithelial cell is encircled by the tight junction complex to form a continuous seal that separates the apical and the basolateral membrane components. Hence, loss of the integrity of cell monolayers results in a loss of the barrier effect in the intestinal epithelium. Agents such as invading bacteria would then come into contact with the immune system, triggering an inflammation response.

### Example 3. Deacidification of Grapefruit and Lemon Juice

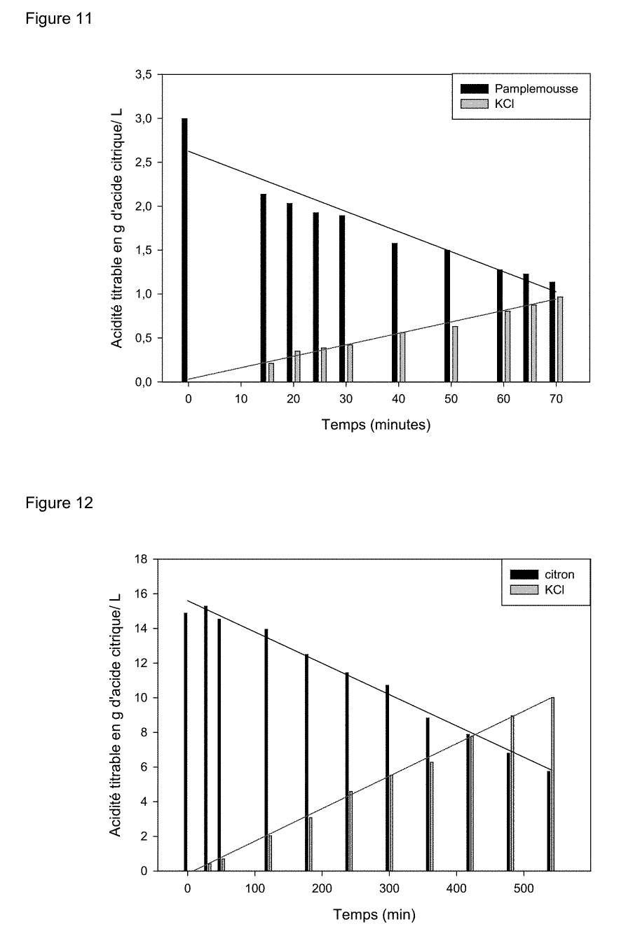

The same methodology was applied to grapefruit juice and produced up to 62% deacidification in 70 minutes (Table 9), while lemon juice was also deacidified up to 62% but in 540 minutes (Table 10). These results, also shown in FIGS. 11 & 12 support the presumption that this technology can be used on a large variety of acid juices to render them easier for consumption without addition of sugar. As well, in the case of grapefruit or lemon juice, this reduction in titratable acidity may also provides less side effects such as stomach irritation, or gastric ulcers.

### Organic Acid Content:

The concentrations of individual organic acid were determined for the ED2 MB configuration. Major organic acids in lemon juice are citric acid and malic acid, whereas the major organic acid in grapefruit juice is citric acid with very low levels of malic acid.

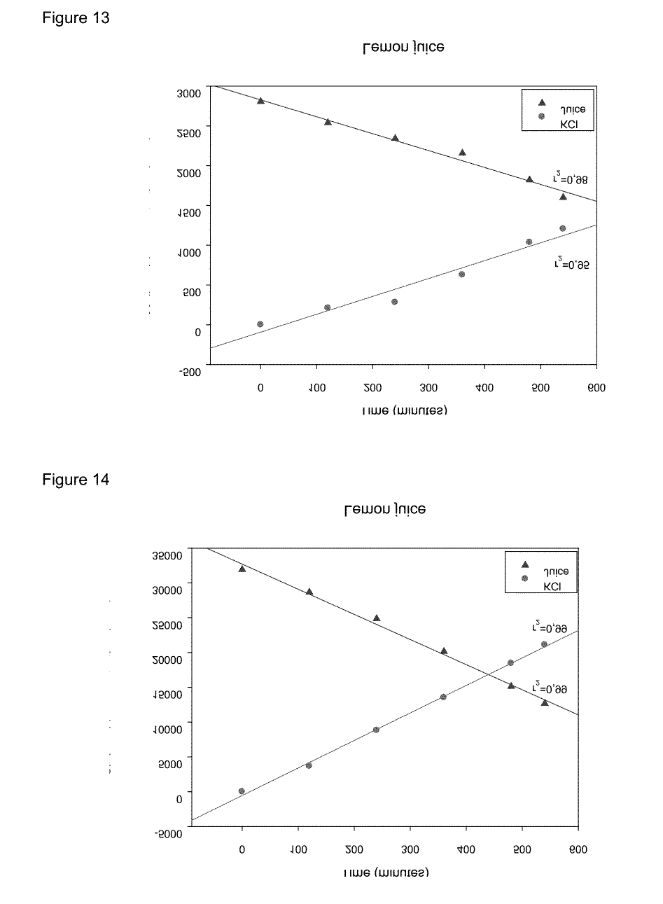

For lemon juice (FIGS. 13 & 14), the level of citric acid decreased at a rate of 36.13 ppm/min in lemon juice. After more than ten (10) hours of treatment, the level of malic acid in the juice was reduced to about 50% (FIG. 13), whereas the level of citric acid was reduced by more than 50% after only about seven hours of treatment (FIG. 14).

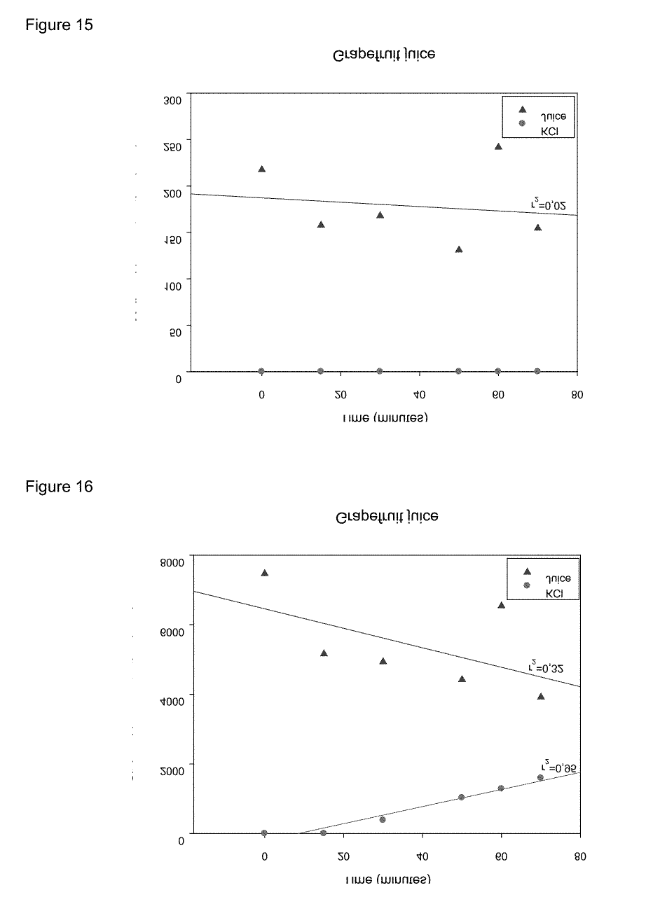

For grapefruit juice (FIGS. 14, 15), the concentration of malic acid remained unaffected throughout the treatment because of no migration (FIG. 14), probably because the initial concentration of malic acid is too low to migrate compared to citric acid. In contrast, citric acid migrated to about 60% of its initial concentration in about 80 minutes (FIG. 15).

### CONCLUSION

This study shows very promising results since deacidified cranberry juice decreased the loss of integrity of the epithelial cell barrier by 56% compared to raw cranberry juice. However, to obtain this positive benefit, a deacidification level of 20% or more is necessary (particularly 37%), since a 19% deacidification level did not protect the in vitro integrity of tight junction complexes. Furthermore, the process of deacidification preserved all other healthy compounds in the cranberry juice, such as anthocyanins and PACs. However, after digestion the anthocyanins disappeared and PAC polymers were depolymerised.

To our knowledge, this is the first time that the deacidification of a cranberry juice, or the decrease in organic acid content of a food, was demonstrated to positively affect the integrity of the intestinal barrier. Consequently, the high antioxidant content and low concentration of malic and citric acids in this improved functional juice, obtained using a green technology, would reduce or eliminate intestinal inflammation and undesirable side effects resulting from regular consumption of cranberry juice. In addition, deacidification of cranberry juice makes healthy compounds from cranberries easily available to the consumer. The market for such a new functional juice is estimated at more than 1.2 million dollars per year for a volume of 750,000 L.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

All patents, patent applications and publications mentioned in this specification are herein incorporated by reference to the same extent as if each independent patent, patent application, or publication was specifically and individually indicated to be incorporated by reference.

TABLE 9
Grapefruit Juice, Brix = 7.65

| | | | | | | | | | | | | | titrable | | titrable | | |
|---|---|---|---|---|---|---|---|---|---|---|---|---|---|---|---|---|---|
| | | | | | | | | | | | | | acidity | | acidity | | |
| | | | | NaCl | | | Juice | | | KCl | | | Juice | | KCl | | |
| | Sample | | | Conduct. | | Temp | Conduct | | Temp | Conduct | | Temp | pH | | pH | | % |
| Time | taken | U (V) | I (A) | (mS) | pH | (° C.) | (mS) | pH | (° C.) | (mS) | pH | (° C.) | 8.2 | g/L eq | 8.2 | g/L eq | deacid |
| 0 | Non | 0 | 0 | 22.72 | 3.72 | 22.9 | 1.483 | 3.4 | 16.9 | 2.316 | 3.67 | 23.2 | | | | 0 | 0 |
| 0 | Yes | 10 | 0.3 | | | | 1.451 | 3.4 | 17.3 | 2.305 | 3.67 | 23.6 | 4.28 | 2.996 | 0 | 0 | 0 |
| 15 | Yes | 10 | 0.31 | | | | 2.278 | 3.48 | 26 | 3.81 | 2.53 | 26.3 | 3.05 | 2.135 | 0.3 | 0.21 | 28.74 |
| 20 | Yes | 10 | 0.31 | | | | 2.304 | 3.5 | 28.2 | 4.026 | 2.47 | 27 | 2.9 | 2.03 | 0.5 | 0.35 | 32.24 |
| 25 | Yes | 10 | 0.31 | | | | 2.285 | 3.54 | 27.6 | 4.206 | 2.44 | 27.7 | 2.75 | 1.925 | 0.55 | 0.385 | 35.75 |
| 30 | Yes | 10 | 0.31 | | | | 2.294 | 361 | 2.94 | 4.323 | 2.39 | 28.2 | 2.7 | 1.89 | 0.6 | 0.42 | 36.92 |
| 40 | Yes | 10 | 0.31 | | | | 2.291 | 3.65 | 29.2 | 4.598 | 2.33 | 29.3 | 2.25 | 1.575 | 0.8 | 0.56 | 47.43 |
| 50 | Yes | 10 | 0.3 | | | | 2.325 | 3.73 | 30.2 | 4.75 | 2.31 | 30.2 | 2.14 | 1.498 | 0.9 | 0.63 | 50 |
| 60 | Yes | 10 | 0.3 | | | | 2.339 | 3.82 | 31.9 | 4.88 | 2.28 | 31 | 1.82 | 1.274 | 1.15 | 0.805 | 57.48 |
| 65 | Yes | 10 | 0.3 | | | | 2.263 | 3.86 | 31.4 | 4.93 | 2.28 | 31.4 | 1.75 | 1.225 | 1.25 | 0.875 | 59.11 |
| 70 | Yes | 10 | 0.29 | 27.43 | 8.34 | 30.8 | 2.261 | 3.9 | 31.7 | 4.98 | 2.25 | 31.4 | 1.62 | 1.134 | 1.38 | 0.966 | 62.15 |

TABLE 10
Lemon juice, °Brix = 7.2

| | | | | | | | | | | | | | Titrable | | titrable | | |
|---|---|---|---|---|---|---|---|---|---|---|---|---|---|---|---|---|---|
| | | | | | | | | | | | | | acidity | | acidity | | |
| | | | | NaCl | | | Juice | | | KCl | | | Juice | | KCl | | |
| | Sample | | | conduct | | Temp | Conduct | | Temp | Conduct | | Temp | pH | | pH | | % |
| Time | taken ? | U (V) | I (A) | (mS) | pH | (° C.) | (mS) | pH | (° C.) | (mS) | pH | (° C.) | 8.2 | g/L eq | 8.2 | g/L eq | deacid |
| 0 | Non | 0 | 0 | 21.49 | 5.07 | 21 | 3.257 | 2.51 | 12.4 | 2.282 | 3.72 | 20.6 | | | | | |
| 0 | Yes | 10 | 0.31 | | | | 3.555 | 2.48 | 20.7 | 2.336 | 3.42 | 21 | 21.25 | 14.875 | 0 | 0 | |
| 30 | Yes | 10 | 0.36 | | | | 4.211 | 2.4 | 27.8 | 3.509 | 2.55 | 27.6 | 21.82 | 15.274 | 0.6 | 0.42 | |
| 50 | Yes | 10 | 0.38 | | | | 4.36 | 2.41 | 30.3 | 3.923 | 2.41 | 30.2 | 20.75 | 14.525 | 1 | 0.7 | 4.90 |
| 120 | Yes | 10 | 0.39 | | | | 4.446 | 2.45 | 34.1 | 4.9 | 2.21 | 33.9 | 19.91 | 13.937 | 2.91 | 2.037 | 8.75 |
| 180 | Yes | 10 | 0.4 | | | | 4.406 | 2.5 | 35.2 | 5.37 | 2.13 | 35.1 | 17.85 | 12.495 | 4.4 | 3.08 | 18.19 |
| 240 | Yes | 10 | 0.4 | | | | 4.334 | 2.55 | 35.7 | 5.68 | 2.08 | 35.6 | 16.35 | 11.445 | 6.55 | 4.585 | 25.069 |
| 300 | Yes | 10 | 0.4 | | | | 4.257 | 2.6 | 36.1 | 6.01 | 2.05 | 36 | 15.3 | 10.71 | 7.9 | 5.53 | 29.88 |
| 360 | Yes | 10 | 0.4 | | | | 4.161 | 2.66 | 36.3 | 6.28 | 2.02 | 36.1 | 12.6 | 8.82 | 8.97 | 6.279 | 42.25 |
| 420 | Yes | 10 | 0.4 | | | | 4.065 | 2.75 | 2.75 | 6.52 | 2.75 | 36.3 | 11.25 | 7.875 | 11.1 | 7.77 | 48.44 |
| 480 | Yes | 10 | 0.4 | | | | 3.965 | 2.85 | 2.85 | 6.74 | 2.85 | 36.4 | 9.7 | 6.79 | 12.8 | 8.96 | 55.54 |
| 540 | Yes | 10 | 0.39 | | | | 3.864 | 2.97 | 2.97 | 6.91 | 2.97 | 36.4 | 8.2 | 5.74 | 14.3 | 10.01 | 62.42 |

NaCl flowrate = 800 mL/min;
KCl and juice flowrate = 450 mL/min

### REFERENCES

1 I. Vasileiou, A. Katsargyris, S. Theocharis and C. Giaginis, Nutr. Res., 2013,33, 595-607.
2 R. Raz, B. Chazan and M. Dan, Clin. Infect. Dis., 2004, 38, 1413-1419.
3 A. B. Howell, J. D. Reed, C. G. Krueger, R. Winterbottom, D. G. Cunningham and M. Leahy, Phytochemistry, 2005,66, 2281-2291.
4 A. B. Howell, H. Botto, C. Combescure, A.-B. Blanc-Potard, L. Gausa, T. Matsumoto, P. Tenke, A. Sotto and J.-P. Lavigne, BMC Infect. Dis., 2010, 10, 94.
5 D. A. Wing, P. J. Rumney, C. W. Preslicka and J. H. Chung, J. Urol., 2008, 180, 1367-1372.
6 C. Khoo and M. Falk, Cranberry Polyphenols: Effects on Cardiovascular Risk Factors, 2014.
7 E. I. Weiss, R. Lev-Dor, N. Sharon and I. Ofek, Crit. Rev. Food Sci. Nutr., 2002,42, 285-292.
8 M. E. T. McMurdo, L. Y. Bissett, R. J. G. Price, G. Phillips and I. K. Crombie, Age Ageing, 2005, 34, 256-261.
9 L. Bazinet, S. Brianceau, P. Dube and Y. Desjardins, Sep. Purif. Technol., 2012,87, 31-39.
10 A. C. Hulme, J. Exp. Bot., 1951, 2, 298-315.
11 G. Shui and L. P. Leong, J. Chromatogr. A, 2002,977, 89-96.
12 F. Chinnici, U. Spinabelli, C. Riponi and A. Amati, J. Food Compos. Anal., 2005,18, 121-130.
13 E. Husson, M. Araya-Farias, A. Gagné and L. Bazinet, J. Memb. Sci., 2013,448, 114-124.
14 D. Labbé and L. Bazinet, J. Memb. Sci., 2006, 275, 220-228.
15 E. Vera, J. Sandeaux, F. Persin, G. Pourcelly, M. Dornier, G. Piombo and J. Ruales, J. Food Eng., 2007, 78, 1439-1445.
16 E. Vera, J. Ruales, M. Dornier, J. Sandeaux, F. Persin, G. Pourcelly, F. Vaillant and M. Reynes, J. Food Eng., 2003,59, 361-367.
17 E. Rozoy, L. Boudesocque and L. Bazinet, J. Agric. Food Chem., 2015,63, 642-651.
18 A. J. Melendez-Martinez, I. M. Vicario and F. J. Heredia, Food Qual. Prefer., 2005,16, 471-478.
19 R. E. Wrolstad, J. Food Sci., 2004, 69, C419-C425.
20 R. C. Khanal, L. R. Howard, C. R. Brownmiller and R. L. Prior, J. Food Sci., 2009, 74, H52-H58.
21 Y. A. L. Waterhouse, Curr. Protoc. Food Anal. Chem., 2002, 1-8.
22 D. A. Cowan and J. H. Brown, Ind. Eng. Chem., 1959, 51, 1445-1448.
23 H. J. Lee and S. H. Moon, J. Colloid Interface Sci., 2005,287, 597-603.
24 M. A. Marietta, Hepatology, 1985, 5, 165.
25 P. Flores, P. Hellin and J. Fenoll, Food Chem., 2012,132, 1049-1054.
26 V. Galli and C. Barbas, J. Chromatogr. A, 2004, 1032, 299-304.
27 L. Bazinet, C. Cossec, H. Gaudreau and Y. Desjardins, J. Agric. Food Chem., 2009, 57, 10245-10251.
28 P. M. Davidson and T. M. Taylor, Food Microbiology: Fundamentals and Frontiers, Third Edition, American Society of Microbiology, 2007.
Bazinet, L., Brianceau, S., Dube, P., & Desjardins, Y. (2012). Evolution of cranberry juice physico-chemical parameters during phenolic antioxidant enrichment by electrodialysis with filtration membrane. Separation and Purification Technology, 87, 31-39.
Bermudez-Soto, M., Tomasbarberan, F., & Garciaconesa, M. (2007). Stability of polyphenols in chokeberry (Aronia melanocarpa) subjected to in vitro gastric and pancreatic digestion. Food Chemistry, 102(3), 865-874.
Cao, G., Alessio, H. M., & Cutler, R. G. (1993). Oxygen-radical absorbance capacity assay for antioxidants. Free Radical Biology and Medicine, 14(3), 303-311.
Chen, G.-L., Chen, S.-G., Zhao, Y.-Y., Luo, C.-X., Li, J., & Gao, Y.-Q. (2014). Total phenolic contents of 33 fruits and their antioxidant capacities before and after in vitro digestion. Industrial Crops and Products, 57, 150-157.
Daugherty, A. L., & Mrsny, R. J. (1999). Regulation of the intestinal epithelial paracellular barrier. Pharmaceutical Science & Technology Today, 2(7), 281-287.
Davies, M. G., & Hagen, P. (1997). Systemic inflammatory response syndrome. British Journal of Surgery, 84(7), 920-935.
Deprez, S., Mila, I., Huneau, J.-F., Tome, D., & Scalbert, A. (2001). Transport of proanthocyanidin dimer, trimer, and polymer across monolayers of human intestinal epithelial Caco-2 cells. Antioxidants and Redox Signaling, 3(6), 957-967.
Ferguson, L. R., Shelling, A. N., Browning, B. L., Huebner, C., & Petermann, I. (2007). Genes, diet and inflammatory bowel disease. Mutation Research, 622(1-2), 70-83.
Fernandes, I., Faria, A., Calhau, C., de Freitas, V., & Mateus, N. (2014). Bioavailability of anthocyanins and derivatives. Journal of Functional Foods, 7, 54-66.
Fiocchi, C. (2003). More answers and more questions in inflammatory bowel disease. Current Opinion in Gastroenterology, 19(4), 325-326.
Froment, D. P. H., Molitoris, B. A., Buddington, B., Miller, N., & Alfrey, A. C. (1989). Site and mechanism of enhanced gastrointestinal absorption of aluminum by citrate. Kidney Int, 36(6), 978-984.
Grant, M., & Leone-Bay, A. (2012). Peptide therapeutics: it's all in the delivery. Therapeutic Delivery, 3(8), 981-996.
Gu, L., House, S. E., Rooney, L., & Prior, R. L. (2007). Sorghum bran in the diet dose dependently increased the excretion of catechins and microbial-derived phenolic acids in female rats. Journal of Agricultural and Food Chemistry, 55(13), 5326-5334.
Holzer, P. (2015). Acid-sensing ion channels in gastrointestinal function. Neuropharmacology, 94, 72-79.
Husson, E., Araya-Farias, M., Gagné, A., & Bazinet, L. (2013). Selective anthocyanins enrichment of cranberry juice by electrodialysis with filtration membrane: Influence of membranes characteristics. Journal of Membrane Science, 448, 114-124.
Jepson, R. G., Williams, G., & Craig, J. C. (2012). Cranberries for preventing urinary tract infections. The Cochrane Database of Systematic Reviews, 10, CD001321.
Khanal, R. C., Howard, L. R., Brownmiller, C. R., & Prior, R. L. (2009). Influence of extrusion processing on procyanidin composition and total anthocyanin contents of blueberry pomace. Journal of Food Science, 74(2), H52-H58.
Khoo, C., & Falk, M. (2014). Polyphenols in Human Health and Disease. Polyphenols in Human Health and Disease. Elsevier.
Kress, M., & Waldmann, R. (2006). Acid sensing ionic channels. Current Topics in Membranes, 57, 241-276.
Krueger, C. G., Chesmore, N., Chen, X., Parker, J., Khoo, C., Marais, J. P. J., . . . Reed, J. D. (2016).
Critical reevaluation of the 4-(dimethylamino)cinnamaldehyde assay: Cranberry proanthocyanidin standard is superior to procyanidin A2 dimer for accurate quantification of proanthocyanidins in cranberry products. Journal of Functional Foods, 22, 13-19.
Kumamoto, M., Sonda, T., Nagayama, K., & Tabata, M. (2001). Effects of pH and metal ions on antioxidative activities of catechins. Bioscience, Biotechnology, and Biochemistry, 65(1), 126-32.
Marietta, M. A. (1985). The merck index. An encyclopedia of chemicals, drugs, and biologicals. Tenth Edition. Martha Windholz, Editor; Susan Budavari, Co-Editor; Rosemary F. Blumetti, Associate Editor, Elizabeth S. Otterbein, Assistant Editor. 2,067 pp. (including tables and in. Hepatology, 5(1), 165.
McGhie, T. K., & Walton, M. C. (2007). The bioavailability and absorption of anthocyanins: towards a better understanding. Molecular Nutrition & Food Research, 51(6), 702-713.
McMurdo, M. E. T., Bissett, L. Y., Price, R. J. G., Phillips, G., & Crombie, I. K. (2005). Does ingestion of cranberry juice reduce symptomatic urinary tract infections in older people in hospital? A double-blind, placebo-controlled trial. Age and Ageing, 34(3), 256-261.
Mikhaylin, S., Nikonenko, V., Pourcelly, G., & Bazinet, L. (2014). Intensification of demineralization process and decrease in scaling by application of pulsed electric field with short pulse/pause conditions. Journal of Membrane Science, 468, 389-399.
Neto, C. C. (2007). Cranberry and blueberry: evidence for protective effects against cancer and vascular diseases. Molecular Nutrition & Food Research, 51(6), 652-664.
Noach, A. B. J., Kurosaki, Y., Blom-Roosemalen, M. C. M., de Boer, A. G., & Breimer, D. D. (1993). Cell-polarity dependent effect of chelation on the paracellular permeability of confluent Caco-2 cell monolayers. International Journal of Pharmaceutics, 90(3), 229-237.
Nolan, C. R., Califano, J. R., & Butzin, C. A. (1990). Influence of calcium acetate or calcium citrate on intestinal aluminium absorption. Kidney Int, 38(5), 937-941.
Ou, K., & Gu, L. (2014). Absorption and metabolism of proanthocyanidins. Journal of Functional Foods, 7, 43-53.
Plumb, G. W., De Pascual-Teresa, S., Santos-Buelga, C., Cheynier, V., & Williamson, G. (1998). Antioxidant properties of catechins and proanthocyanidins: effect of polymerisation, galloylation and glycosylation. Free Radical Research, 29(4), 351-8.
Raz, R., Chazan, B., & Dan, M. (2004). Cranberry Juice and Urinary Tract Infection. Clinical Infectious Diseases, 38 (10), 1413-1419.
Romanov-Michailidis, F., Viton, F., Fumeaux, R., Leveques, A., Actis-Goretta, L., Rein, M., . . . Barron, D.
(2012). Epicatechin B-ring conjugates: First enantioselective synthesis and evidence for their occurrence in human biological fluids. Organic Letters, 14(15), 3902-3905.
Romier-Crouzet, B., Van De Walle, J., During, A., Joly, A., Rousseau, C., Henry, O., . . . Schneider, Y.-J. (2009). Inhibition of inflammatory mediators by polyphenolic plant extracts in human intestinal Caco-2 cells. Food and Chemical Toxicology, 47(6), 1221-1230.
Rozoy, E., Boudesocque, L., & Bazinet, L. (2015). Deacidification of Cranberry Juice by Electrodialysis with Bipolar Membranes. Journal of Agricultural and Food Chemistry, 63(2), 642-651.
Ruiz, P. A., & Haller, D. (2006). Functional diversity of flavonoids in the inhibition of the proinflammatory NF-κB, IRF, and Akt signaling pathways in murine intestinal epithelial cells. The Journal of Nutrition, 136(3), 664-671.
Sánchez-Patán, F., Cueva, C., Monagas, M., Walton, G. E., Gibson, G. R., Martin-Álvarez, P. J., Moreno-Arribas, M. V., & Bartolome, B. (2012). Gut microbial catabolism of grape seed flavan-3-ols by human faecal microbiota. Targetted analysis of precursor compounds, intermediate metabolites and end-products. Food Chemistry, 131(1), 337-347.
Sergent, T., Piront, N., Meurice, J., Toussaint, O., & Schneider, Y.-J. (2010). Anti-inflammatory effects of dietary phenolic compounds in an in vitro model of inflamed human intestinal epithelium. Chemico-Biological Interactions, 188(3), 659-667.
Serre, E., Rozoy, E., Pedneault, K., Lacour, S., & Bazinet, L. (2016). Deacidification of cranberry juice by electrodialysis: Impact of membrane types and configurations on acid migration and juice physicochemical characteristics. Separation and Purification Technology, 163, 228-237.
Sharma, A., & Zhou, W. (2011). A stability study of green tea catechins during the biscuit making process. Food Chemistry, 126(2), 568-573.
Spencer, J. P. E., Chaudry, F., Pannala, A. S., Srai, S. K., Debnam, E., & Rice-Evans, C. (2000). Decomposition of cocoa procyanidins in the gastric milieu. Biochemical and Biophysical Research Communications, 272(1), 236-241.
Steen, K. H., Steen, A. E., & Reeh, P. W. (1995). A dominant role of acid pH in inflammatory excitation and sensitization of nociceptors in rat skin, in vitro. The Journal of Neuroscience, 15(5), 3982-3989.
Sun, H., Chow, E. C., Liu, S., Du, Y., & Pang, K. S. (2008). The Caco-2 cell monolayer: usefulness and limitations. Expert Opinion on Drug Metabolism & Toxicology, 4(4), 395-411.
Tomita, M., Hayashi, M., & Awazu, S. (1996). Absorption-enhancing mechanism of EDTA, caprate, and decanoylcarnitine in Caco-2 cells. Journal of Pharmaceutical Sciences, 85(6), 608-611.
Vachon, P. H., & Beaulieu, J.-F. (1992). Transient mosaic patterns of morphological and functional differentiation in the Caco-2 cell line. Gastroenterology, 103(2), 414-423.
Vasileiou, I., Katsargyris, A., Theocharis, S., & Giaginis, C. (2013). Current clinical status on the preventive effects of cranberry consumption against urinary tract infections. Nutrition Research, 33(8), 595-607.
Vera, E., Sandeaux, J., Persin, F., Pourcelly, G., Dornier, M., & Ruales, J. (2007). Deacidification of clarified tropical fruit juices by electrodialysis. Part I. Influence of operating conditions on the process performances. Journal of Food Engineering, 78(4), 1427-1438.
Vermeulen, M. A. R., de Jong, J., Vaessen, M. J., van Leeuwen, P. A. M., & Houdijk, A. P. J. (2011). Glutamate reduces experimental intestinal hyperpermeability and facilitates glutamine support of gut integrity. World Journal of Gastroenterology: WJG, 17(12), 1569.
Versantvoort, C. H. M., Oomen, A. G., Van de Kamp, E., Rompelberg, C. J. M., & Sips, A. J. A. M. (2005). Applicability of an in vitro digestion model in assessing the bioaccessibility of mycotoxins from food. Food and Chemical Toxicology: An International Journal Published for the British Industrial Biological Research Association, 43(1), 31-40.
Wada, L., & Ou, B. (2002). Antioxidant activity and phenolic content of Oregon caneberries. Journal of Agricultural and Food Chemistry, 50(12), 3495-3500.
Waldner, M. J., & Neurath, M. F. (2009). Novel cytokine-targeted therapies and intestinal inflammation. Current Opinion in Pharmacology, 9(6), 702-7.
Waterhouse, Y. A. L. (2002). Determination of Total Phenolics. Current Protocols in Food Analytical Chemistry, 1-8.
Welling, S. H., Hubálek, F., Jacobsen, J., Brayden, D. J., Rahbek, U. L., & Buckley, S. T. (2014). The role of citric acid in oral peptide and protein formulations: relationship between calcium chelation and proteolysis inhibition. European Journal of Pharmaceutics and Biopharmaceutics: Official Journal of Arbeitsgemeinschaft Für Pharmazeutische Verfahrenstechnik e.V, 86(3), 544-51.
Wemmie, J. A., Price, M. P., & Welsh, M. J. (2006). Acid-sensing ion channels: advances, questions and therapeutic opportunities. Trends in Neurosciences, 29(10), 578-586.
Wing, D. A., Rumney, P. J., Preslicka, C. W., & Chung, J. H. (2008). Daily cranberry juice for the prevention of asymptomatic bacteriuria in pregnancy: a randomized, controlled pilot study. The Journal of Urology, 180(4), 1367-1372.
Wrolstad, R. E. (2004). Anthocyanin pigments—Bioactivity and coloring properties. Journal of Food Science, 69(5), C419-C425.
Zimnicka, A. M., Ivy, K., & Kaplan, J. H. (2011). Acquisition of dietary copper: a role for anion transporters in intestinal apical copper uptake. American Journal of Physiology-Cell Physiology, 300(3), C588-0599.

1. A deacidified juice having a malic acid or citric acid concentration decreased by at least about 20% compared with said juice untreated and having an original malic acid or citric acid concentration.

2. The juice of claim 1, wherein said malic acid is 40% lower than in said untreated juice.

3. The juice of claim 1, wherein said malic acid is 60% lower than in said untreated juice.

4. (canceled)

5. The juice of claim 1, being selected from: lemon, lime, passion fruit, apple, cherries, loganberry, plum, prune, pomegranate, pear, blackberry, raspberry, strawberry, tangerine blueberry, currant, blackcurrant, pineapple, kiwi, peach, apricot, tomato, orange, acai and cranberry juice.

6. The juice of claim 3, being selected from: cranberry and lemon juice.

7. (canceled)

8. The juice of claim 1, wherein said citric acid is 40% lower than in said untreated juice.

9. The juice of claim 1, wherein said citric acid is 50% lower than in said untreated juice.

10-11. (canceled)

12. The juice of claim 9, being selected from: cranberry, grapefruit and lemon juice.

13. The juice of claim 12, being a cranberry juice and having a citric acid concentration lower than about 16,200 mg/L.

14. The juice of claim 13, wherein said citric acid is lower than about 12,000 mg/L.

15. (canceled)

16. The juice of claim 13, wherein said citric acid is lower than about 8,000 mg/L.

17. (canceled)

18. The juice of claim 6, being a cranberry juice and having a malic acid concentration lower than about 10,600 mg/L.

19-20. (canceled)

21. The juice of claim 18, wherein said malic acid is lower than about 6,000 mg/L.

22-23. (canceled)

24. The juice of claim 2, being a cranberry juice with a deacidification level of at least about 20% wherein said level is calculated with the equation (1):
deacidification level (in %)=(1−TA₍ₜ₎/TA₍ₜ₌₀₎)*100 (1)
wherein TA is the titratable acidity.

25-27. (canceled)

28. The juice of claim 1, being a cranberry juice with a titrable acidity lower than 7 g/L of citric acid monohydrate equivalents, when assessed using AOAC method no. 942.15 (2005).

29-31. (canceled)

32. A process for treating and/or preventing urinary tract infections without provoking serious intestinal side effects, comprising administering a deacidifed cranberry juice as defined in claim 1.

33. A process for deacidifying a juice high in malic and/or citric acid concentration, comprising the steps of:
subjecting said juice to electrodialysis with one anion-exchange membrane (AEM) having a cathode side and an anode side, and juxtaposed on both sides with a bipolar (BP) membrane according to scheme I: [symbol]
for a length of time sufficient to decrease titrable acidity by at least about 20%.

34-41. (canceled)

42. A method for diminishing undesirable intestinal effects of a juice having an original citric acid or malic acid concentration, comprising lowering said original citric acid or malic acid concentration by at least about 20%.

43-45. (canceled)

46. The juice of claim 1, wherein said juice has an original pH, and said treated juice has a treated pH, and said treated juice pH has less than 0.5 unit difference from said original pH.

47-48. (canceled)

49. The method of claim 42, wherein said undesirable intestinal effects comprise an alteration of integrity of epithelial cell junctions or intestinal cell integrity.
